(19) United States
(12) Patent Application Publication
(10) Pub. No.: US 2014/0114385 A1
(43) Pub. Date: Apr. 24, 2014

(54) DEVICES, SYSTEMS AND METHODS FOR MODULATION OF THE NERVOUS SYSTEM

(71) Applicants: Harold Nijhuis, Zeist (NL); David S. Wood, Mountain View, CA (US); Daniel M. Brounstein, San Francisco, CA (US)

(72) Inventors: Harold Nijhuis, Zeist (NL); David S. Wood, Mountain View, CA (US); Daniel M. Brounstein, San Francisco, CA (US)

(73) Assignee: Spinal Modulation, Inc., Menlo Park, CA (US)

(21) Appl. No.: 13/839,324

(22) Filed: Mar. 15, 2013

Related U.S. Application Data
(60) Provisional application No. 61/621,948, filed on Apr. 9, 2012.
(60) Provisional application No. 61/712,731, filed on Oct. 11, 2012.

(51) Int. Cl. A61N 1/05

(52) U.S. Cl. CPC A61N 1/0551
USPC 607/117

(57) ABSTRACT

Devices, systems and methods are provided to modulate portions of neural tissue of the nervous system, such as portions of the central nervous system or portions of the peripheral nervous system. In some embodiments, the systems and devices of the present invention are used to stimulate one or more dorsal root ganglia, dorsal roots, dorsal root entry zones, or portions thereof, while minimizing or excluding undesired stimulation of other tissues, such as surrounding or nearby tissues, ventral root and portions of the anatomy associated with body regions which are not targeted for treatment. In other embodiments, the systems and devices are used to stimulate portions of the peripheral nervous system. In some embodiments, the modulation generates a massaging sensation, particularly when stimulating neural tissue on particular spinal levels, such as L2 or L3.

### CROSS-REFERENCES TO RELATED APPLICATIONS

This application claims priority under 35 U.S.C. 119(e) to U.S. Provisional Patent Application No. 61/621,948, entitled “Devices, Systems and Methods for Modulation of the Nervous System”, filed Apr. 9, 2012, and U.S. Provisional Patent Application No. 61/712,731, entitled “Selective Stimulation for the Treatment of Pain in the Lower Torso”, filed Oct. 11, 2012, both of which are incorporated herein by reference for all purposes.

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND OF THE INVENTION

Pain of any type is the most common reason for physician consultation in the United States, prompting half of all Americans to seek medical care annually. It is a major symptom in many medical conditions, significantly interfering with a person's quality of life and general functioning. Diagnosis is based on characterizing pain in various ways, according to duration, intensity, type (dull, burning, throbbing or stabbing), source, or location in body. Usually if pain stops without treatment or responds to simple measures such as resting or taking an analgesic, it is then called ‘acute’ pain. But it may also become intractable and develop into a condition called chronic pain in which pain is no longer considered a symptom but an illness by itself.

It has been reported that more than 1.5 billion people worldwide suffer from chronic pain and that approximately 3-4.5% of the global population suffers from neuropathic pain, pain resulting from damage or disease affecting the somatosensory system. Chronic pain can be mild or excruciating, episodic or continuous, merely inconvenient or totally incapacitating.

Of suffers of chronic pain, low back pain is particularly prevalent and causes significant debilitation. When asked about four common types of pain, respondents of a National Institute of Health Statistics survey indicated that low back pain was the most common (27%), followed by severe headache or migraine pain (15%), neck pain (15%) and facial ache or pain (4%). Back pain is the leading cause of disability in Americans under 45 years old. More than 26 million Americans between the ages of 20-64 experience frequent back pain. Adults with low back pain are often in worse physical and mental health than people who do not have low back pain: 28% of adults with low back pain report limited activity due to a chronic condition, as compared to 10% of adults who do not have low back pain. Also, adults reporting low back pain were three times as likely to be in fair or poor health and more than four times as likely to experience serious psychological distress as people without low back pain.

A significant portion of patients suffering from low back pain are experiencing referred pain due to the Thoracolumbar Junction Syndrome (also known as TLJ Syndrome, Maigne syndrome, posterior ramus syndrome and dorsal ramus syndrome). Thoracolumbar Junction Syndrome is defined by a dysfunction of the thoracolumbar junction (TLJ) referring pain in the whole or part of the territory of the corresponding dermatomes (eg. T11 to L1 or L2). Depending on the branch involved, the pain could refer to the low back (cutaneous dorsal rami), to the groin (subcostal or iliohypogastric nerve) or in the lateral aspect of the hip (lateral cutaneous rami of the subcostal or iliohypogastric nerve, or cluneal nerve). All combinations of these clinical presentations are possible with one, two or three involved territories.

Low back pain is the most frequently encountered pain complaint in the TLJ syndrome. The pain is distributed in the dermatomes of T11, T12, L1 or L2. Because the limits of these dermatomes are ill defined, due to overlapping and anastomosis, the pain is usually spread in the lateral part of the low back without corresponding exactly to a specific dermatome. Rarely, the pain is bilateral; more often, it is unilateral. The pain is usually acute, of less than 2 or 3 months duration, often appearing after a false rotatory movement of the trunk, prolonged strenuous posture, lifting and occasionally, without any obvious precipitating factors.

Since the dermatomes covering the groin are T12, L1 and L2, groin pain is easily related to a TLJ origin. Groin pain may accompany low back pain or be an isolated complaint. The pain may sometimes be located above the groin, in the T10 or T11 dermatomes, depending on the involved level of the TLJ.

The third feature of the TLJ syndrome is pain over the lateral aspect of the hip. It is a referred pain in the territory of the lateral cutaneous branch of either the iliohypogastric or the subcostal nerve, or an extension of the cluneal nerve.

Each of the different pain syndromes characterizing the TLJ syndrome can appear in isolation or in combination in a given patient. The most common cause is a dysfunction at T10/T11, T11/T12 or T12/L1. This area is vulnerable as a large percentage of the total rotation in the spine comes from the TLJ. As such when other segments become restricted the TLJ may be over utilized in rotation either acutely or chronically resulting in dysfunction within the motion segment. The nature of this dysfunction remains unknown, although the involvement of either the facets or the disc is very likely. Some other causes are possible, although very rare, such as a disc herniation or a collapse of the vertebral body of T11, L2 or L1 referring pain only in the low back

The treatment of the TLJ syndrome is at first the treatment of the TLJ itself. The TLJ syndrome can be responsive to spinal manipulative therapy. Manipulation is a forced movement applied to a joint within the anatomic limits. This movement is characterized by a cracking sound due to a vacuum phenomenon as the facets separate. The vacuum phenomenon, or cavitation, makes the separation of the articular surfaces very sudden, even more so than the movement which initiated it. Thus, the cavitation appears as a motion accelerator, which could play a role by stretching hypertonic muscles. This is true not only for the TLJ but also for any part of the spine. The separation of the facets could also unblock the motion segment. Manipulation may also act on the disc. This could alter the load transmission through the disc, thought to be one of the factors transforming a pain free degenerated disc into a painful one.

If manipulative treatment is unsuccessful or contraindicated for a particular patient, such as in an elderly patient where osteoarthritis is likely, facet injections can be performed. A facet injection is an injection of a steroid in the facet joints which are located in the back area the spinal bony structure. The steroid injected reduces the inflammation and/or swelling of tissue in the joint space. This may in turn reduce pain, and other symptoms caused by inflammation or irritation of the joint and surrounding structures. The effects of facet injections tend to be temporary, providing relief for several days or three to six months.

If facet injections are successful, longer relief may be provided by a facet rhizotomy, a procedure that uses an electrical current to destroy the nerve fibers carrying pain signals to the brain. Using a local anesthetic and x-ray guidance, a needle with an electrode at the tip is placed alongside the small nerves to the facet joint. The electrode is then heated, with a technology called radiofrequency, to deaden these nerves that carry pain signals to the brain. The effects of facet rhizotomy are also temporary, providing relief for two months to 1 year.

Improved treatments are desired to provide more lasting relief to patients suffering from both TLJ syndrome and other ailments causing chronic pain, particularly low back pain. Likewise, improved treatments are desired to treat patients who are poor candidates for existing treatments or have not received adequate pain relief. At least some of these objectives will be met by the present invention.

For other types of chronic pain, nerve stimulation has been utilized. For example, the application of specific electrical energy to the spinal cord has been actively practiced since the 1960s for the purpose of managing pain. It is known that application of an electrical field to spinal nervous tissue can effectively mask certain types of pain transmitted from regions of the body associated with the stimulated nervous tissue. Such masking is known as paresthesia, a subjective sensation of numbness or tingling in the afflicted bodily regions. Such electrical stimulation of the spinal cord, once known as dorsal column stimulation, is now referred to as spinal cord stimulation or SCS.

Alternatively, in some cases, peripheral neurostimulation or combination of spinal and peripheral stimulation is applied. In the case of peripheral stimulation, one or more electrodes are subcutaneously placed in the area where the pain is localized and are subcutaneously connected with an implantable generator. The electric energy produced acts on the peripheral nerves in order to reduce pain.

For evaluating the therapeutic result of nerve stimulation, a testing period of a few days takes place. Typically, pain relief of at least 50% and consent is required before permanent system is implanted. The programming of the system is done telemetrically with a remote control. Programming selects and applies various stimulation parameter combinations that provide the optimal therapeutic result. The patient also is equipped with a remote control to turn off, activate and turn up and down the stimulator. In the case that there is no satisfactory result, the electrode(s) placed are withdrawn and removed. It is a fully reversible therapy, meaning that every neuromodulation system can at any time be removed in case of necessity.

Although conventional SCS and peripheral stimulation systems have effectively relieved pain in some patients, these systems have a number of drawbacks. To begin, in conventional SCS, the lead is positioned upon the spinal cord dura layer near the midline of the spinal cord. The electrodes stimulate a wide portion of the spinal cord and associated spinal nervous tissue. Significant energy is utilized to penetrate the dura layer and cerebral spinal fluid to activate fibers in the spinal column extending within the posterior side of the spinal cord. Sensory spinal nervous tissue, or nervous tissue from the dorsal nerve roots, transmit pain signals. Therefore, such stimulation is intended to block the transmission of pain signals to the brain with the production of a tingling sensation (paresthesia) that masks the patient's sensation of pain. However, excessive tingling may be considered undesirable. Further, the energy also typically penetrates the anterior side of the spinal cord, stimulating the ventral horns, and consequently the ventral roots extending within the anterior side of the spinal cord. Motor spinal nervous tissue, or nervous tissue from ventral nerve roots, transmits muscle/motor control signals. Therefore, electrical stimulation by the lead often causes undesirable stimulation of the motor nerves in addition to the sensory spinal nervous tissue. The result is undesirable muscle contraction.

Because the electrodes span several levels and because they stimulate medial to spinal root entry points, the generated stimulation energy stimulates or is applied to more than one type of nerve tissue on more than one level. Moreover, these and other conventional, non-specific stimulation systems also apply stimulation energy to the spinal cord and to other neural tissue beyond the intended stimulation targets. As used herein, non-specific stimulation refers to the fact that the stimulation energy is provided to multiple spinal levels including the nerves and the spinal cord generally and indiscriminately. This is the case even with the use of programmable electrode configurations wherein only a subset of the electrodes are used for stimulation. In fact, even if the epidural electrode is reduced in size to simply stimulate only one level, that electrode will apply stimulation energy non-specifically and indiscriminately (i.e. to many or all nerve fibers and other tissues) within the range of the applied energy.

One of the drawbacks of conventional peripheral stimulation is the large lead migration rates. Often soft tissue anchors are inadequate in keeping the lead in the desired position, primarily due to the movement of tissues in the periphery which can place mechanical forces on the lead instigating migration. This has been observed in several locations within the body from the lower extremities, to the occipital region as well as areas in the axial trunk.

Therefore, improved stimulation systems, devices and methods are desired that enable more precise and effective delivery of stimulation energy. In addition, improved stimulation systems, devices and methods are desired that provide treatment with minimal undesired side effects and loss of efficacy over time, such as due to migration or device failure. At least some of these objectives will be met by the present invention.

### SUMMARY OF THE INVENTION

Aspects of the present disclosure provide devices, systems, and methods for modulating portions of the nervous system.

In a first aspect of the present invention, a method is provided of treating pain in a patient. In some embodiments, the method comprises positioning a lead having at least one electrode so that at least one of the at least one electrodes is near a dorsal root ganglion associated with the pain, and selecting at least one stimulation parameter so that energy is provided to the at least one of the at least one electrodes which results in selective stimulation of at least a portion of the dorsal root ganglion, wherein the selective stimulation causes a massaging sensation which treats the pain.

In some embodiments, positioning the lead further comprises positioning the lead so that at least one of the at least one electrodes is near a mixed spinal nerve, a dorsal ramus and/or a ventral ramus associated with the dorsal root ganglion. In such embodiments, selecting at least one stimulation parameter may further comprise selecting at least one stimulation parameter so that energy is provided to at least one of the electrodes which results in selective stimulation of the mixed spinal nerve, the dorsal ramus, and/or the ventral ramus.

In some embodiments, the dorsal root ganglion is disposed on spinal level T10, T11, T12, L1, L2, L3, L4 or L5. In particular, in some embodiments, the dorsal root ganglion is disposed on spinal level L2 or L3. Likewise, in some embodiments, the pain is associated with a thoracolumbar junction of the patient. Similarly, the pain may be located in a low back of the patient.

In some embodiments, the method further comprises selecting at least one stimulation parameter so that the selective stimulation causes undesired muscle contraction prior to selecting the at least one stimulation parameter so that the selective stimulation causes the massaging sensation.

In some embodiments, selecting at least one stimulation parameter comprises selecting a frequency of at least 16 Hz. For example, in some embodiments, selecting at least one stimulation parameter comprises selecting a frequency in the range of approximately 20-50 Hz. Or, in other embodiments, selecting at least one stimulation parameter comprises selecting a frequency in the range of approximately 20 Hz. In some embodiments, the method further comprises selecting a frequency of less than or equal to 10 Hz so that a motor contraction is generated and then increasing the frequency to the frequency of at least 16 Hz to cause the massaging sensation.

In another aspect of the present invention, a method is provided of treating a condition of a patient comprising advancing a lead having at least one electrode into an epidural space in an antegrade direction, laterally away from a midline of a spinal cord and through a foramen so that at least one of the at least one electrodes is positioned near a peripheral nerve associated with the condition, and selecting at least one stimulation parameter so that energy is provided to the at least one of the at least one electrodes which results in selective stimulation of at least a portion of the peripheral nerve, wherein the selective stimulation treats the condition.

In some embodiments, the selective stimulation causes a massaging sensation. In some embodiments, the peripheral nerve comprises a mixed spinal nerve. For example, in some embodiments, the peripheral nerve comprises a dorsal ramus. For example, in some embodiments, the peripheral nerve comprises a lateral branch, a medial branch, or an intermediate branch. In other embodiments, the peripheral nerve comprises a ventral ramus.

In some embodiments, the method further comprises selecting at least one stimulation parameter so that energy is provided to at least one electrode which results in selective stimulation of at least a portion of a dorsal root ganglion disposed within the foramen.

In some embodiments, the foramen is disposed on spinal level T10, T11, T12, L1, L2, L3, L4 or L5. In particular, the foramen may be disposed on spinal level L2 or L3. In some embodiments, the condition comprises pain associated with a thoracolumbar junction of the patient. In some embodiments, the condition comprises pain located in a low back of the patient.

In some embodiments, selecting at least one stimulation parameter comprises selecting a frequency of at least 16 Hz. In particular, in some embodiments, selecting at least one stimulation parameter comprises selecting a frequency in the range of approximately 20-50 Hz. Likewise, in some embodiments, selecting at least one stimulation parameter comprises selecting a frequency in the range of approximately 20 Hz.

In some embodiments, the method further comprises selecting a frequency of less than or equal to 10 Hz so that a motor contraction is generated and then increasing the frequency to the frequency of at least 16 Hz to treats the condition by generating a massaging sensation.

Other objects and advantages of the present invention will become apparent from the detailed description to follow, together with the accompanying drawings.

### BRIEF DESCRIPTION OF THE DRAWINGS

FIG. 1 illustrates an embodiment of a stimulation system of the present invention.

FIG. 2 illustrates example placement of the leads of the embodiment of FIG. 1 within the patient anatomy.

FIG. 3 illustrates a cross-sectional view of an individual spinal level showing a lead of the stimulation system positioned near a target dorsal root ganglion.

FIG. 4 illustrates a pair of spinal nerves extending from the spinal cord at a spinal level.

FIG. 5 illustrates an example distribution of spinal nerve branches within a patient.

FIGS. 6A-6C illustrate example areas of referred pain.

FIG. 7 illustrates spinal nerve branches extending from the vertebral column, including an embodiment of a lead positioned so as to selectively stimulate a dorsal root ganglion.

FIG. 8 illustrates an embodiment of optional lead placement wherein the distal end of the lead is extended through the foramen so as to position at least one electrode near the mixed nerve.

FIG. 9 illustrates an embodiment wherein the lead is advanced so that at least one electrode is positioned along the medial branch of the dorsal ramus.

FIG. 10 illustrates an embodiment wherein the lead is advanced further through the foramen so that at least one electrode is positioned along a portion of the ventral ramus.

FIGS. 11A-11B illustrate a lumbar plexus and an embodiment of a lead of the present invention positioned so as to selectively stimulate a portion of the lumbar plexus.

FIG. 12 illustrates an embodiment of a lead of the present invention positioned so as to selectively stimulate the trigeminal ganglion.

FIG. 13 illustrates an embodiment of a lead of the present invention positioned so as to selectively stimulate a portion of a cervical plexus.

FIGS. 14, 15A-15B illustrate a brachial plexus and an embodiment of a lead of the present invention positioned so as to selectively stimulate a portion of the brachial plexus.

FIG. 16A illustrates a cross-sectional view of the thoracic cavity and associated spinal anatomy.

FIG. 16B illustrates a lead of the present invention positioned so as to selectively stimulate a portion of an intercostal nerve.

FIGS. 17A-17B illustrate a sacral plexus and an embodiment of a lead of the present invention positioned so as to selectively stimulate a portion of the sacral plexus.

FIGS. 18A-18D illustrate an embodiment of a lead and delivery system.

FIG. 19 illustrates an embodiment of a sheath advanced over the shaft of a lead until a portion of its distal end abuts the distal tip of the lead.

FIG. 20 illustrates an embodiment of a stylet disposed within a lead so that extension of the lead and stylet through the sheath bends or directs the lead.

FIG. 21 illustrates an embodiment of an assembled sheath/lead/stylet that is advanced within the epidural space toward a DRG.

FIG. 22 illustrates the lead/stylet of FIG. 21 able to be advanced beyond the distal end of the sheath along a nerve root.

### DETAILED DESCRIPTION OF THE INVENTION

The present invention provides for targeted treatment of a variety of conditions with minimal deleterious side effects, such as undesired stimulation of unaffected body regions. This is achieved by directly neuromodulating a target anatomy associated with the condition while minimizing or excluding undesired neuromodulation of other anatomies. In most embodiments, neuromodulation comprises stimulation, however it may be appreciated that neuromodulation may include a variety of forms of altering or modulating nerve activity by delivering electrical or pharmaceutical agents directly to a target area. For illustrative purposes, descriptions herein will be provided in terms of stimulation and stimulation parameters, however, it may be appreciated that such descriptions are not so limited and may include any form of neuromodulation and neuromodulation parameters.

In some embodiments, the systems and devices are used to stimulate portions of neural tissue of the central nervous system, wherein the central nervous system includes the spinal cord and the pairs of nerves along the spinal cord which are known as spinal nerves. The spinal nerves include both dorsal and ventral roots which fuse to create a mixed nerve which is part of the peripheral nervous system. At least one dorsal root ganglion (DRG) is disposed along each dorsal root prior to the point of mixing. Thus, the neural tissue of the central nervous system is considered to include the dorsal root ganglions and exclude the portion of the nervous system beyond the dorsal root ganglions, such as the mixed nerves of the peripheral nervous system. In some embodiments, the systems and devices of the present invention are used to stimulate one or more dorsal root ganglia, dorsal roots, dorsal root entry zones, or portions thereof, while minimizing or excluding undesired stimulation of other tissues, such as surrounding or nearby tissues, ventral root and portions of the anatomy associated with body regions which are not targeted for treatment. In other embodiments, the systems and devices are used to stimulate portions of the peripheral nervous system. And, in still other embodiments, the systems and devices are used to stimulate other tissues including those described and illustrated herein. In some embodiments, example methods and devices for selectively stimulating target tissues are provided in U.S. Pat. Nos. 7,502,651; 7,337,005; 7,337,006; 7,450,993; 7,580,753; 7,447,546, each incorporated herein by reference for all purposes.

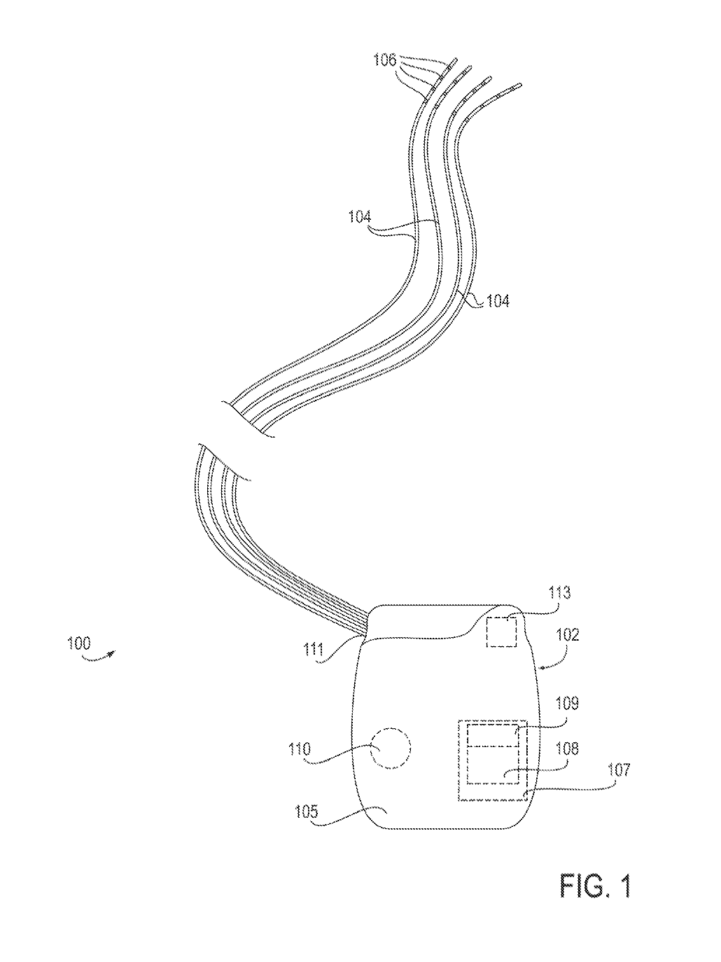

FIG. 1 illustrates an embodiment of an implantable stimulation system 100 for treatment of a patient. The system 100 includes an implantable pulse generator (IPG) 102 and at least one lead 104 connectable thereto. In preferred embodiments, the system 100 includes four leads 104, as shown, however any number of leads 104 may be used including one, two, three, four, five, six, seven, eight, up to 58 or more. Each lead 104 includes at least one electrode 106. In preferred embodiments, each lead 104 includes four electrodes 106, as shown, however any number of electrodes 106 may be used including one, two, three, four five, six, seven, eight, nine, ten, eleven, twelve, thirteen, fourteen, fifteen, sixteen or more. Each electrode can be configured as off, anode or cathode. In some embodiments, even though each lead and electrode is independently configurable, at any given time the software ensures only one lead is stimulating at any time. In other embodiments, more than one lead is stimulating at any time, or stimulation by the leads is staggered or overlapping.

Referring again to FIG. 1, the IPG 102 includes electronic circuitry 107 as well as an antenna 113 and power supply 110, e.g., a battery, such as a rechargeable or non-rechargeable battery, so that once programmed and turned on, the IPG 102 can operate independently of external hardware. In some embodiments, the electronic circuitry 107 includes a processor 109 and programmable stimulation information in memory 108.

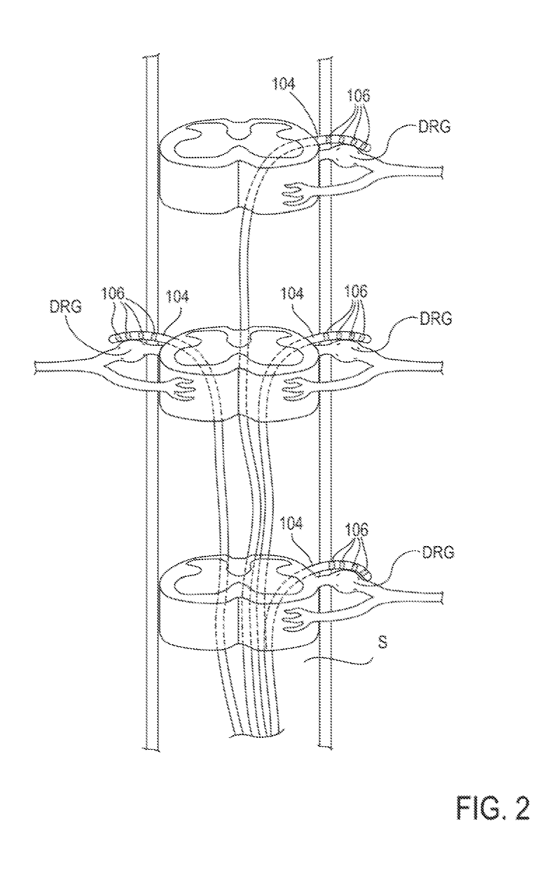

The implantable stimulation system 100 can be used to stimulate a variety of anatomical locations within a patient's body. In some embodiments, the system 100 is used to stimulate one or more dorsal roots, particularly one or more dorsal root ganglions. FIG. 2 illustrates example placement of the leads 104 of the embodiment of FIG. 1 within the patient anatomy. In this example, each lead 104 is individually advanced within the spinal column S in an antegrade direction. Each lead 104 has a distal end which is guidable toward a target DRG and positionable so that its electrodes 106 are in proximity to the target DRG. In particular, FIG. 2 illustrates the stimulation of four DRGs, each DRG stimulated by one lead 104. These four DRGs are located on three levels, wherein two DRGs are stimulated on the same level. It may be appreciated that any number of DRGs and any combination of DRGs may be stimulated with the stimulation system 100 of the present invention. It may also be appreciated that more than one lead 104 may be positioned so as to stimulate an individual DRG and one lead 104 may be positioned so as to stimulate more than one DRG.

In the embodiment of FIG. 2, each lead 104 is positionable so that its electrodes 106 are able to selectively stimulate the desired target, such as the target DRG, either due to position, electrode configuration, electrode shape, electric field shape, electrode size, stimulation signal parameters, stimulation signal, pattern or algorithm, or any combination of these. Used herein, selective stimulation is stimulation of the target anatomy with little, no or imperceptible stimulation of unwanted anatomies. Such selective stimulation stimulates the targeted neural tissue while excluding untargeted tissue, such as surrounding or nearby tissue. In some embodiments, the stimulation energy is delivered to the targeted neural tissue so that the energy dissipates or attenuates beyond the targeted tissue or region to a level insufficient to stimulate modulate or influence such untargeted tissue. In some embodiments, selective stimulation of the DRG describes stimulation of the DRG only, stimulation of the DRG with imperceptible or no stimulation of surrounding tissue, or stimulation of the DRG without stimulation of the ventral root, such as wherein the stimulation signal has an energy below an energy threshold for stimulating a ventral root associated with the target dorsal root while the lead is so positioned. Examples of methods and devices to achieve such selective stimulation of the dorsal root and/or DRG are provided in U.S. patent application Ser. No. 12/607,009, entitled “Selective Stimulation Systems and Signal Parameters for Medical Conditions”, incorporated herein by reference for all purposes.

It may be appreciated that selective stimulation or neuromodulation concepts described herein may be applied in a number of different configurations. Unilateral (on or in one root ganglion on a level), bi-lateral (on or in two root ganglion on the same level), unilevel (one or more root ganglion on the same level) or multi-level (at least one root ganglion is stimulated on each of two or more levels) or combinations of the above including stimulation of a portion of the sympathetic nervous system and one or more dorsal root ganglia associated with the neural activity or transmission of that portion of the sympathetic nervous system. As such, embodiments of the present invention may be used to create a wide variety of stimulation control schemes, individually or overlapping, to create and provide zones of treatment.

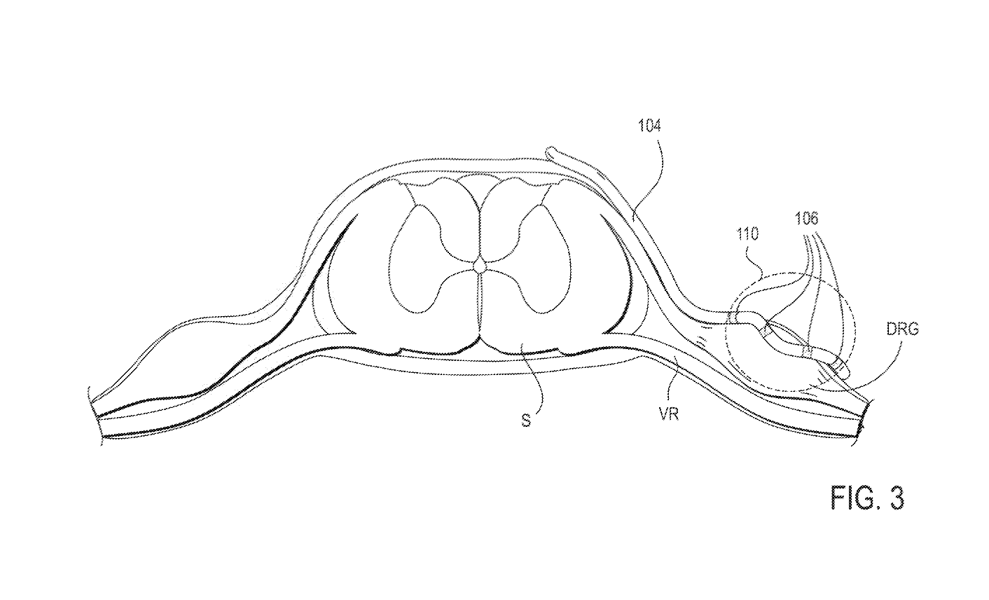

FIG. 3 illustrates an example cross-sectional view of an individual spinal level showing a lead 104 of the stimulation system 100 positioned on a target DRG. The lead 104 is advanced along the spinal cord S to the appropriate spinal level wherein the lead 104 is advanced laterally toward the target DRG. In some instances, the lead 104 is advanced through or partially through a foramen. At least one, some or all of the electrodes 106 are positioned on, near, about, adjacent or in proximity to the DRG. In preferred embodiments, the lead 104 is positioned so that the electrodes 106 are disposed along a surface of the DRG opposite to the ventral root VR, as illustrated in FIG. 3. It may be appreciated that the surface of the DRG opposite the ventral root VR may be diametrically opposed to portions of the ventral root VR but is not so limited. Such a surface may reside along a variety of areas of the DRG which are separated from the ventral root VR by a distance.

In order to position the lead 104 in such close proximity to the DRG, the lead 104 is appropriately sized and configured to maneuver through the anatomy. In some embodiments, such maneuvering includes atraumatic epidural advancement along the spinal cord S, through a sharp curve toward a DRG, and optionally through a foramen wherein the distal end of the lead 104 is configured to then reside in close proximity to a small target such as the DRG. Consequently, the lead 104 is significantly smaller and more easily maneuverable than conventional spinal cord stimulator leads. Example leads and delivery systems for delivering the leads to a target such as the DRG are provided in U.S. patent application Ser. No. 12/687,737, entitled “Stimulation Leads, Delivery Systems and Methods of Use”, incorporated herein by reference for all purposes.

Referring again to FIG. 3, the electrodes 106 provide a stimulation region which is selective to the target anatomy. In this embodiment, the stimulation region is indicated by dashed line 110, wherein the DRG receives stimulation energy within the stimulation region and the ventral root VR does not as it is outside of the stimulation region. Selective stimulation of the DRG is achieved with the choice of the size of the electrode(s), the shape of the electrode(s), the position of the electrode(s), the stimulation signal, pattern or algorithm, or any combination of these. Such selective stimulation stimulates the targeted neural tissue while excluding untargeted tissue, such as surrounding or nearby tissue. In some embodiments, the stimulation energy is delivered to the targeted neural tissue so that the energy dissipates or attenuates beyond the targeted tissue or region to a level insufficient to stimulate modulate or influence such untargeted tissue. In particular, selective stimulation of tissues, such as the dorsal root, DRG, or portions thereof, exclude stimulation of the ventral root wherein the stimulation signal has an energy below an energy threshold for stimulating a ventral root associated with the target dorsal root while the lead is so positioned. Examples of methods and devices to achieve such selective stimulation of the dorsal root and/or DRG are provided in U.S. patent application Ser. No. 12/607,009, entitled “Selective Stimulation Systems and Signal Parameters for Medical Conditions”, incorporated herein by reference for all purposes.

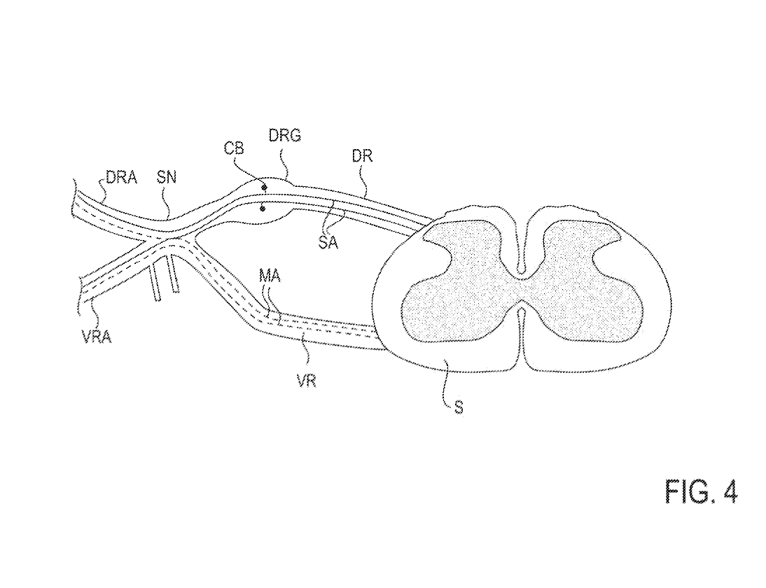

FIG. 4 provides an additional illustration of a pair of spinal nerves extending from the spinal cord S at a spinal level. The pairs are comprised of a dorsal root DR and a ventral root VR. The dorsal root DR carries afferent sensory axons SA (indicated by solid lines) extending from cell bodies CB which are located in the dorsal root ganglion DRG. The ventral root VR carries efferent motor axons MA (indicated by dashed lines). The dorsal root DR and ventral root VR fuse forming a mixed spinal nerve SN which emerges from the spinal column through an opening (intervertebral foramen) between adjacent vertebrae. This is true for all spinal nerves except for the first spinal nerve pair, which emerges between the occipital bone and the atlas (the first vertebra). Thus the cervical nerves are numbered by the vertebra below, except C8, which exists below C7 and above T1. The thoracic, lumbar, and sacral nerves are then numbered by the vertebra above. In the case of a lumbarized S1 vertebra (aka L6) or a sacralized L5 vertebra, the nerves are typically still counted to L5 and the next nerve is S1. Humans have 31 pairs of spinal nerves, each roughly corresponding to a segment of the vertebral column: 8 cervical spinal nerve pairs (C1-C8), 12 thoracic pairs (T1-T12), 5 lumbar pairs (L1-L5), 5 sacral pairs (S1-S5), and 1 coccygeal pair.

The mixed spinal nerve SN carries motor, sensory, and autonomic signals between the spinal cord and the body. Outside the vertebral column, the nerve divides into branches. The dorsal ramus DRA contains nerves that serve the dorsal portions of the trunk carrying visceral motor, somatic motor, and somatic sensory information to and from the skin and muscles of the back (epaxial muscles). The ventral ramus VRA contains nerves that serve the remaining ventral parts of the trunk and the upper and lower limbs (hypaxial muscles) carrying visceral motor, somatic motor, and sensory information to and from the ventrolateral body surface, structures in the body wall, and the limbs. The meningeal branches (recurrent meningeal or sinuvertebral nerves) branch from the spinal nerve and re-enter the intervertebral foramen to serve the ligaments, dura, blood vessels, intervertebral discs, facet joints, and periosteum of the vertebrae. The rami communicantes (white ramus and gray ramus) contain autonomic nerves that serve visceral functions carrying visceral motor and sensory information to and from the visceral organs.

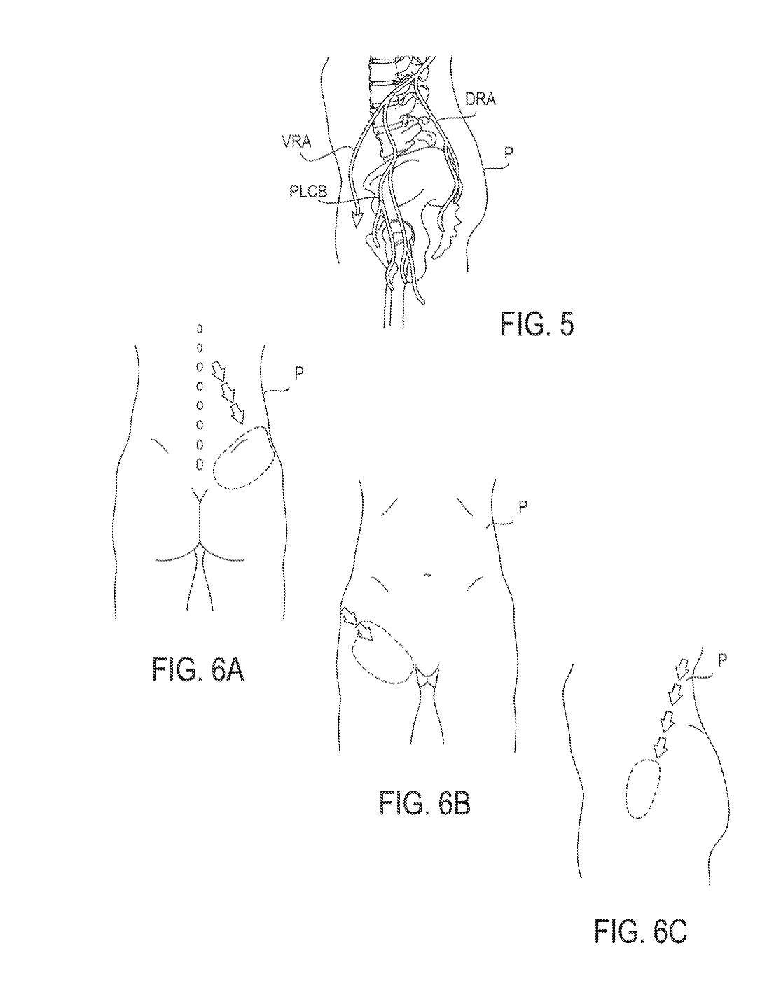

FIG. 5 illustrates an example distribution of spinal nerve branches, such as at T12 or L1, within a patient P. The T12 and L1 spinal nerves emerge at the level of the thoracolumbar junction TLJ and follow a similar course. The dorsal ramus DRA is shown extending posteriorly, down toward the low back, and the ventral ramus VRA is shown extending anteriorly. The ventral ramus VRA supplies the skin of the lower abdomen, the medial aspect of the upper thigh, and the labia majora or the scrotum, the lower part of the rectus abdominis and transversus abdominis muscles, and the pubis. The ventral ramus VRA also branches into a perforating lateral cutaneous branch PLCB which, in this example, emerges above the greater trochanter and supplies the skin of the upper lateral part of the thigh.

Referred pain from the thoracolumbar junction TLJ is felt in the cutaneous distribution of these nerves, the skin and subcutaneous tissues are the site of reflex cellulalgia. However, the pain is felt as deep pain. FIGS. 6A-6C illustrate areas of referred pain, as indicated by shading. FIG. 6A illustrates low back pain (dorsal ramus DRA). FIG. 6B illustrates pseudovisceral pain and groin pain (ventral ramus VRA). FIG. 6C illustrates pseudotrochanteric pain (perforating lateral cutaneous branch PLCB). Usually, the cause of such referred pain is painful minor intervertebral dysfunction of a thoracolumbar junction TLJ segment, most often T12/L1, sometimes T11/T12 or L1/L2 (more rare is the T10/T11 level).

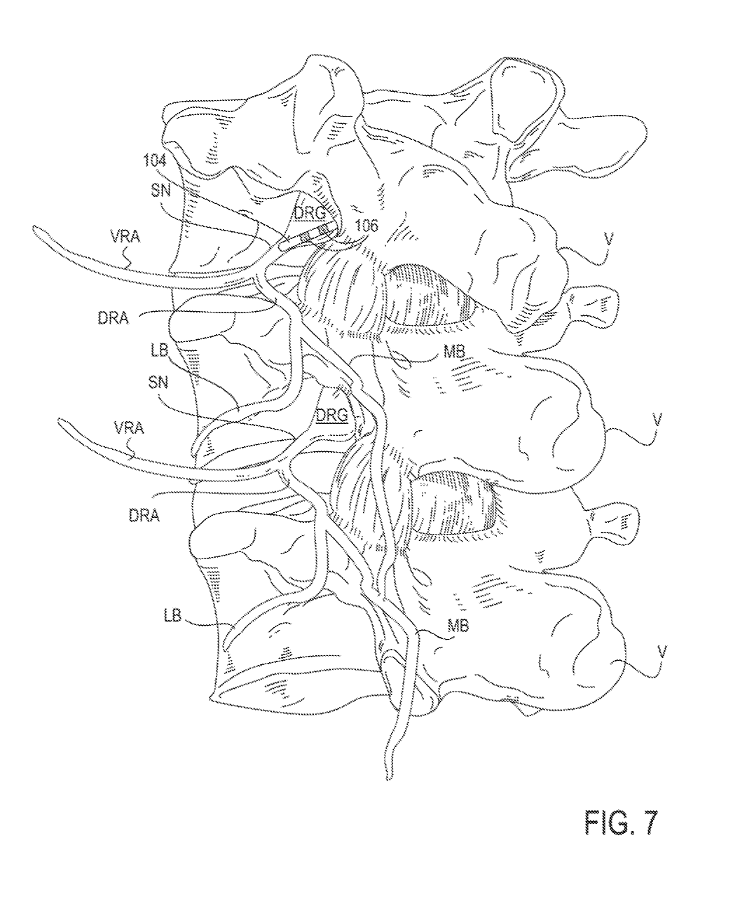

FIG. 7 illustrates spinal nerve branches extending from the vertebral column. Three vertebrae V are illustrated forming a portion of the vertebral column. Each dorsal root ganglion DRG is shown residing within a foramen, an opening between adjacent vertebrae. Beyond the DRG, the mixed spinal nerve SN emerges and divides into branches, the ventral ramus VRA and the dorsal ramus DRA. As shown, each dorsal ramus DRA branches into at least a lateral branch LB and a medial branch MB. The medial branches innervate the facet joints which are often involved in TLJ syndrome and referred pain to the low back.

### Treatment of Pain in the Low Torso

The systems, methods and devices provided herein are able to treat patients suffering from TLJ syndrome or other conditions causing pain in the low torso, such as the low back, hip, groin or other areas. Such systems, methods and devices involve neuromodulating a dorsal root ganglion and/or other neural anatomies to relieve or eliminate the pain sensations. As mentioned previously, in most embodiments, neuromodulation comprises stimulation, however it may be appreciated that neuromodulation may include a variety of forms of altering or modulating nerve activity by delivering electrical or pharmaceutical agents directly to a target area. For illustrative purposes, descriptions herein will be provided in terms of stimulation and stimulation parameters, however, it may be appreciated that such descriptions are not so limited and may include any form of neuromodulation and neuromodulation parameters.

Referring again to FIG. 7, a lead 104 is shown positioned so as to selectively stimulate the DRG. In this embodiment, the lead 104 is positioned so that at least one of the electrodes 106 is adjacent the DRG. Such placement at spinal level L2 or L3 is particularly suitable for treatment of pain in the lower torso, such as pain associated with the thoracolumbar junction TLJ, low back pain, pseudovisceral pain and groin pain, hip pain and pseudotrochanteric pain, to name a few. However, it may be appreciated that such pain can be treated by such placement of leads 104 at spinal levels T10, T11, T12, L1, L2, L3, L4 and/or L5 due to communications between the lumbar spinal nerves. In any case, a variety of stimulation parameters (electrode selection, amplitude, frequency, pulse width) can be selected to most effectively treat the pain. In some embodiments, a massaging sensation is generated. Such a massaging sensation differs from a feeling of paresthesia. In some embodiments, the massaging sensation mimics the feeling of pressure, rubbing, fibrillating or kneading of a muscle. Such a sensation is in contrast to paresthesia which typically has a feeling of burning, prickling, itching, tingling, or “pins and needles” feeling. In some embodiments, the massaging sensation is located in the low back. In particular, in some embodiments, the massaging sensation is felt in the supra-axial muscles, such as the m. multifidus, m. longissimus, and/or m. iliocostalis. It may be appreciated that in some embodiments the massaging sensation is felt in other anatomies, such as the hip or groin. In particular, in some embodiments, the massaging sensation is felt in the infra-axial muscles, such as m. psoas major and/or m. psoas minor It may also be appreciated that, in some embodiments, the massaging sensation is felt in the iliopsoas, m. quadratus lumborum, thoracolumbar fascia, m. intertransversarii lumborum or a combination of these, to name a few. Referring back to FIG. 7, in this embodiment, at least a portion of the dorsal root ganglion is stimulated by at least one electrode 106 disposed along the lead 104 to generate such a massaging sensation in a correlated muscle of the lower torso. Such correlation is dictated by innervation by motor and DRG neurons. Spinal segments of motor neurons innervating a muscle are the same as those of DRG neurons projecting afferent fibers from the muscle. Myotomes of lumbar spinal muscles differ from the segmental level of the lumbar spinal site from which the muscle innervation originates. The segmental innervation patterns of the efferent and afferent nervous system are synchronized, showing a non-metameric pattern. In at least one study, at L5, neurons labeled with fluorescent neurotracer were prominent in DRG L3 for the lamina, L2 for the spinous process, L2 for the back muscle fascia, and L1 for the skin. Dorsal elements are therefore innervated by neurons in more rostral DRG. Muscles originating from the L5 vertebrae are innervated by motor and DRG neurons predominately in the L2 and L3 spinal levels. Thus, DRGs on various spinal levels have differing innervation patterns. Consequently, stimulation of particular DRGs, such as on spinal levels L2 or L3, are able to generate the massaging sensation in particular muscles with stimulation parameter values that differ from those used on other spinal levels. Stereoscopically, the peripheral innervation territory of a lumbar DRG is conical, with the apex at the ganglion and the base circumference located on the dermatome. The lumbar spine itself is involved in the conical innervation territories of DRG.

In some embodiments, frequency of the stimulation signal is adjusted to generate the desired massaging sensation in the patient. For example, in some embodiments, one or more signal parameters are adjusted to produce undesired muscle contractions. This indicates the location of the stimulation and the muscles or body region. that is affected by the stimulation. The one or more signal parameters are then readjusted to produce a massaging sensation instead of the undesired muscle contractions. Typically, stimulation at a frequency of less than or equal to approximately 10 Hz, 5 Hz, 4 Hz, 3 Hz, 2 Hz, 1 Hz, 4-5 Hz, 4-10 Hz generates undesired muscle contraction. In some embodiments, adjustment of the frequency to a value such as approximately at least 16 Hz, 16-100 Hz, 20 Hz, 30, Hz, 40 Hz, 50 Hz, 20-50 Hz, 20-30 Hz, 20-40 Hz, 30-40 Hz, 30-50 Hz, 40-50 Hz generates the massaging sensation which is desired by the patient to treat the pain. In some embodiments, the amplitude of the stimulation signal is approximately less than or equal to 2000 μA, more particularly approximately less than or equal to 1000 μA. In some embodiments, the pulse width of the stimulation signal is approximately 40-300 msec, 200 msec, 300 msec, or 200-300 msec. In some embodiments, pulse width is reduced from a level which provides stimulation sensations in the groin to a level which eliminates stimulation sensations in the groin or reduces such sensations so that they are imperceptible or not noticeable.

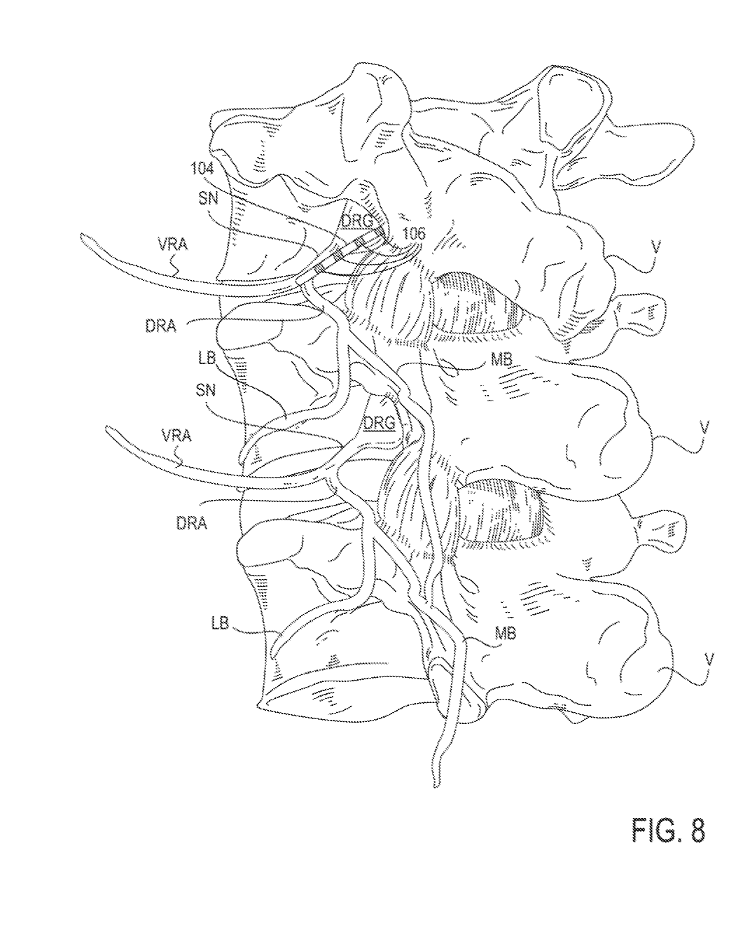

In other embodiments, the lead 104 is positioned so as to selectively stimulate other target tissues, such as at least a portion of the mixed spinal nerve SN, the ventral ramus VRA, the dorsal ramus DRA, the lateral branch LB, the medial branch MB, the intermediate branch IB, and/or the rami communicantes (white ramus and/or gray ramus), to name a few. In some embodiments, such stimulation of other target tissue is achieved in combination with selective stimulation of at least a portion of the associated DRG. Selective stimulation of one or more of such branches at spinal levels T10, T11, T12, L1, L2, L3, L4 and/or L5 are particularly suitable for treatment of pain in the lower torso, such as pain associated with the thoracolumbar junction TLJ. FIG. 8 illustrates an optional lead 104 placement wherein the distal end of the lead 104 is extended through the foramen so as to position at least one electrode near the mixed spinal nerve SN, so as to selectively stimulate the mixed spinal nerve SN. In this embodiment, stimulation parameters (particularly electrode 106 selection) may be varied to selectively stimulate the mixed spinal nerve, the DRG, or both. Such stimulation excludes direct stimulation of the ventral root which would lead to undesired motor effects, such as undesired or uncomfortable muscle contraction. In some instances, when the lead 104 is placed as in FIG. 8, stimulation parameters may also be varied to selectively stimulate portions of the dorsal ramus DRA and/or ventral ramus VRA while excluding direct stimulation of the ventral root. In some embodiments, when stimulating one or a combination of DRG, mixed spinal nerve, dorsal ramus DRA and ventral ramus VRA, a massaging sensation is generated. Such a massaging sensation differs from a feeling of paresthesia as mentioned above. In some embodiments, when stimulating a portion of the dorsal rami, the massaging sensation is located in the low back, such as in the supra-axial muscles, such as m. multifidus, m. longissimus, and/or m. iliocostalis. The m. multifidus in particular is a muscle that is targeted in treating low back pain, i.e. the location of the massaging sensation. The multifidus muscle is comprised of a number of fleshy and tendinous fasciculi which fill up the groove on either side of the spinous processes of the vertebrae, from the sacrum to the axis. The multifidus is a very thin muscle. Deep in the spine, it spans three joint segments and works to stabilize the joints at each segmental level. The stiffness and stability makes each vertebra work more effectively, and reduces the degeneration of the joint structures. It may be appreciated that in some embodiments the massaging sensation is felt in other anatomies, such as the hip or groin. In some embodiments, when stimulating the ventral rami, the massaging sensation is located in the infra-axial muscles, such as m. psoas major and/or m. psoas minor. Muscles originating from the transverse process of the spine, such as m. intertransversarii lumborum or m. quadratus lumborum, are supplied by nerve branches either from the dorsal or ventral rami of spinal nerves and are therefore affected by stimulation of these nerve branches. It may be appreciated that at least a portion of the iliopsoas and/or thoracolumbar fascia can be affected by stimulation of an associated spinal nerve. Thus, when at least a portion of the mixed spinal nerve SN, dorsal ramus DRA or ventral ramus VRA are stimulated, the massaging sensation is caused by stimulation of motor nerves therein. Recall, the mixed spinal nerve SN, dorsal ramus DRA and ventral ramus VRA carry both motor and sensory neurons. Likewise, motor and DRG neurons innervate muscles in the lower torso as described above.

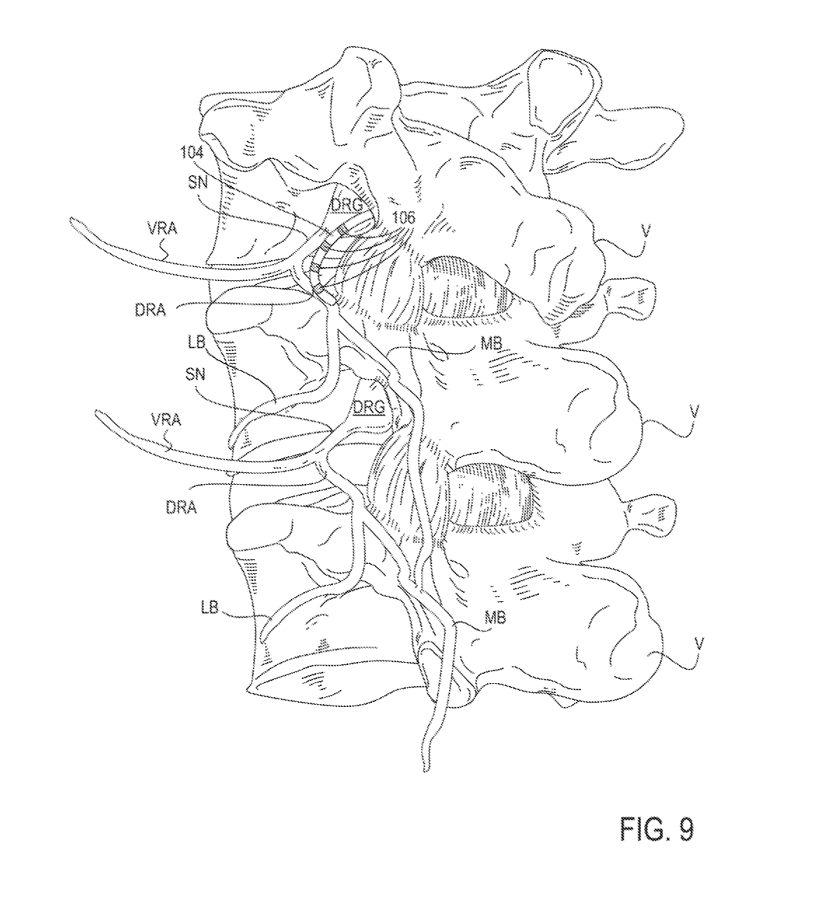

In some embodiments, the lead 104 is advanced further through the foramen so that at least one electrode 106 is positioned along a portion of the dorsal ramus DRA, such as along the lateral branch LB, the intermediate branch IB, and/or the medial branch MB, to name a few. It may be appreciated, that the medial branch MB includes articular branches to the zygopophyseal joints and the periosteum of the vertebral arch. FIG. 9 illustrates an embodiment wherein the lead 104 is advanced so that at least one electrode 106 is positioned along the medial branch MB of the dorsal ramus DRA. Such placement is particularly suitable for treatment of low back pain, particularly back pain associated with the medial branch MB and/or associated with the thoracolumbar junction TLJ. Stimulation parameters can be varied (particularly electrode selection) to selectively stimulate portions of the medial branch MB or any nearby nerve tissue (such as portions of the lateral branch LB, intermediate branch IB or mixed spinal nerve SN, to name a few, without direct stimulation of the ventral nerve. When stimulating one or a combination of these nerve tissues, a massaging sensation is generated. Such a massaging sensation differs from a feeling of paresthesia, as mentioned previously. It may be appreciated that in other embodiments the lead 104 of FIG. 9 is advanced further along the dorsal ramus DRA so as to stimulate particular branches that are further from the foramen.

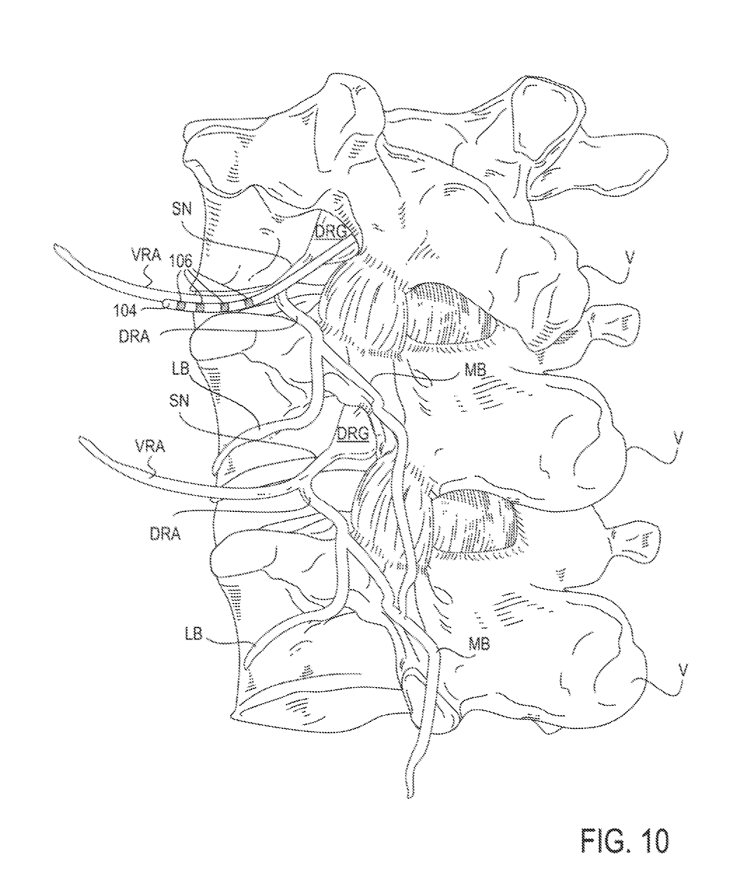

FIG. 10 illustrates an embodiment wherein the lead 104 is advanced further through the foramen so that at least one electrode 106 is positioned along a portion of the ventral ramus VRA. Such placement is particularly suitable for treatment of pseudovisceral pain and groin pain, hip pain and pseudotrochanteric pain, such as when associated with the thoracolumbar junction TLJ. To treat such pain, a variety of stimulation parameters (electrode selection, amplitude, frequency, pulse width) can be selected to most effectively treat the pain. In some embodiments, a massaging sensation is generated. As mentioned, such a massaging sensation differs from a feeling of paresthesia.

Advancement of the lead 104 as in FIG. 9 and FIG. 10 provides stimulation directly to the target spinal anatomies beyond the foramen. In some of these embodiments, frequency of the stimulation signal is adjusted to generate the desired massaging sensation in the patient. Typically, stimulation at a frequency of less than or equal to approximately 10 Hz, 5 Hz, 4 Hz, 3 Hz, 2 Hz, 1 Hz, 4-5 Hz, 4-10 Hz generates undesired muscle contraction. In some embodiments, adjustment of the frequency to a value such as approximately at least 16 Hz, 16-100 Hz, 20 Hz, 30, Hz, 40 Hz, 50 Hz, 20-50 Hz, 20-30 Hz, 20-40 Hz, 30-40 Hz, 30-50 Hz, 40-50 Hz generates the massaging sensation which is desired by the patient to treat the pain. In some embodiments, the amplitude of the stimulation signal is approximately less than or equal to 2000 μA, more particularly approximately less than or equal to 1000 μA. In some embodiments, the pulse width of the stimulation signal is approximately 40-300 msec, 200 msec, 300 msec, or 200-300 msec.

It may be appreciated that, in some embodiments, the massaging sensation and/or pain relief described herein is achievable with the methods, systems and devices described herein in patients having morphologic changes to their nerve anatomy due to chronic pain, disease, various conditions or other factors. In other words, the morphologic changes to the nerve anatomy of such patients causes such patients to react differently than if such patients were normal, healthy and/or pain-free. For example, in some embodiments, the massaging sensation resulting from selective stimulation of a DRG in the area of the TLJ is felt in a patient suffering from TLJ syndrome and is not felt in a patient that is pain-free or is not suffering from TLJ syndrome. Likewise, in some embodiments, the massaging sensation and/or pain relief described herein is achievable using the methods, systems and devices described herein on specific spinal levels wherein such effects are not generated on other spinal levels. In particular, dorsal root ganglions differ in anatomical makeup and/or neural networking depending on spinal level; consequently, differing effects may be achieved. In some embodiments, spinal levels L2 and L3 are particularly suitable for generation of the massaging sensation.

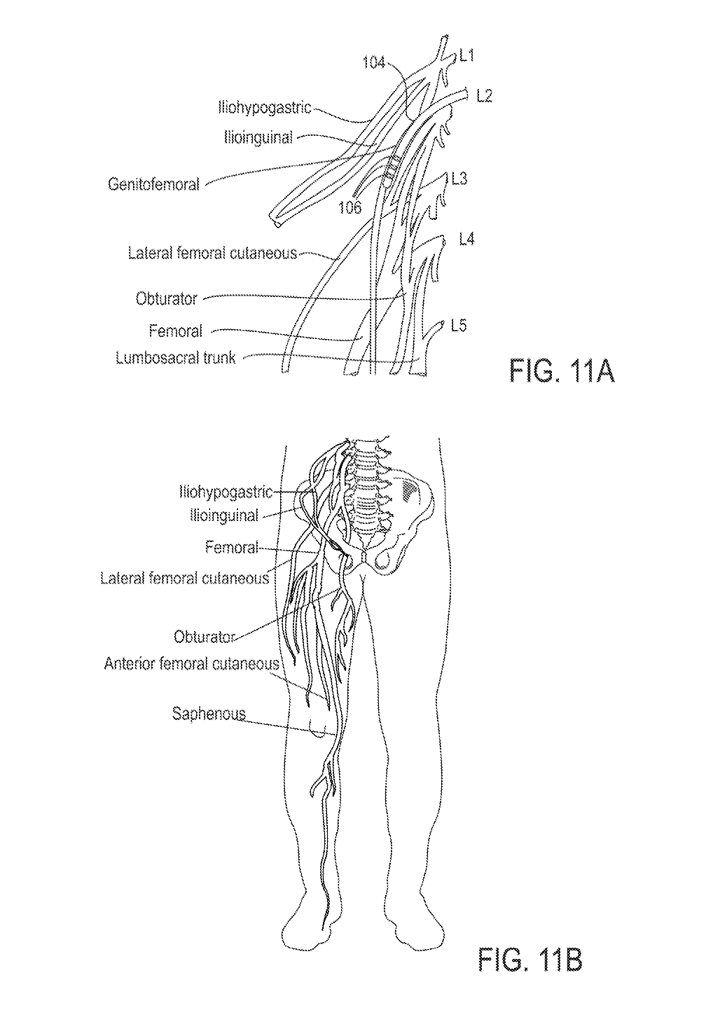

It may be appreciated that the methods, systems and devices may be used to target other peripheral nerves in the low torso or low back area. For example, in some embodiments, the systems and devices of the present invention are used to selectively stimulate portions of the lumbar plexus. Referring to FIGS. 11A-11B, the lumbar plexus is a nervous plexus in the lumbar region of the body which forms part of the lumbosacral plexus. As shown in FIG. 11A, it is formed by the ventral divisions of the first four lumbar nerves (L1-L4) and from contributions of the subcostal nerve (T12), which is the last thoracic nerve. Additionally, the ventral rami of the fourth lumbar nerve pass communicating branches, the lumbosacral trunk, to the sacral plexus. As shown in FIG. 11B, the nerves of the lumbar plexus pass in front of the hip joint and mainly support the anterior part of the thigh. The plexus is formed lateral to the intervertebral foramina and pass through psoas major. Its smaller motor branches are distributed directly to psoas major, while the larger branches leave the muscle at various sites to run obliquely downward through the pelvic area to leave the pelvis under the inguinal ligament, with the exception of the obturator nerve which exits the pelvis through the obturator foramen.

In one embodiment, the lead 104 is advanced from lumbar segment L2 and positioned within the lumbar plexus, as shown in FIG. 11A. Here, the lead 104 is positioned along the genitofemoral nerve. It may be appreciated that the lead 104 may similarly be positioned along any of the nerves of the lumbar plexus. Referring again to FIG. 11A, the lead 104 is positioned so as to selectively stimulate a portion of the lumbar plexus. Selective stimulation is achieved with the choice of the size of the electrode(s), the shape of the electrode(s), the position of the electrode(s), the stimulation signal, pattern or algorithm, or any combination of these. Such selective stimulation stimulates the targeted neural tissue while excluding untargeted tissue, such as surrounding or nearby tissue. In some embodiments, the stimulation energy is delivered to the targeted neural tissue so that the energy dissipates or attenuates beyond the targeted tissue or region to a level insufficient to stimulate modulate or influence such untargeted tissue, such as nerves leading to other regions of the leg. In some embodiments, the methods and devices to achieve such selective stimulation are provided in U.S. patent application Ser. No. 12/607,009, entitled “Selective Stimulation Systems and Signal Parameters for Medical Conditions”, incorporated herein by reference for all purposes. In other embodiments, methods and devices to selectively stimulate any of the nerves of the lumbar plexus are specific to the anatomy of the nerves of the lumbar plexus and/or the condition treated. In particular, the stimulation signal parameters may include a pulse width, frequency, amplitude or a combination of these which selectively stimulates the genitofemoral nerve or any other nerve in the region while excluding or minimizing the stimulation of surrounding or other anatomies.

The lead 104 may be advanced along portions of the lumbar plexus so as to desirably position the electrodes 106 along the target nerve. In order to achieve such positioning, the lead 104 is appropriately sized and configured to maneuver through the local anatomy. Consequently, the lead 104 is significantly smaller and more easily maneuverable than conventional spinal cord stimulator leads. In some embodiments, the leads and delivery systems used to reach such target sites within the anatomy are provided in U.S. patent application Ser. No. 12/687,737, entitled “Stimulation Leads, Delivery Systems and Methods of Use”, incorporated herein by reference for all purposes. In some embodiments, the devices include specialized delivery devices for delivering and positioning the lead near the nerves of the lumbar plexus, such as the genitofemoral nerve (as illustrated) or the iliohypogastric nerve, the ilioinguinal nerve, the lateral femoral cutaneous nerve, the obturator nerve, the femoral nerve or the lumbosacral trunk, to name a few. The lead may also have particular features, such as small diameter and high flexibility which enable desirable placement and reduced migration in the area of the lumbar plexus.

It may be appreciated that the lead 104 may be advanced to portions of the lumbar plexus or other targets within or beyond the lumbar plexus via atraumatic epidural advancement along the spinal cord S and exiting through a lumbar foramen. However, it may also be appreciated that these targets may be reached by other access routes, including direct access through the skin to the target site, such as in directly into the leg. In some embodiments, the target nerve is accessed with the use of a needle under guided visualization, such as guided ultrasound. The lead 104 is advanced through the needle so as to position at least one of the electrodes 106 near or on the portion of the nerve to receive the stimulation. It may be appreciated that in some embodiments, at least one of the electrodes 106 is positioned within the nerve. In some embodiments, the lead 104 is so positioned with the use of an introducer which has a stiffness suitable for advancement through the tissue near the target nerve anatomy. Likewise, in some embodiments, the lead is tunneled through the tissue with the use of one or more dilators. It may also be appreciated that in some embodiments the lead 104 is implanted via conventional surgical methods, or using other endoscopic or laparoscopic techniques.

### Treatment of Discogenic Pain

It may be appreciated that the methods, systems and devices may be utilized on any spinal level and is not limited to the spinal levels associated with TLJ syndrome. It may also be appreciated that the methods, systems and devices may be used to treat other diseases and conditions and is not limited to treating TLJ syndrome or any of the specific areas of pain enumerated herein. For example, the methods, systems and devices may be utilized to treat primary discogenic pain, such as of the lumbar spine. Such pain is often evident in both post-laminotomy syndrome (PLS) and degenerative disc disease (DDD). The principle location of this pain is in the axial distribution of the low back and has often proven to be a challenging target for conventional intraspinal neurostimulation. Typically, the bilateral L2 gray ramus communicans nerves (GRC) are involved in the transmission of discogenic nociceptive pain. Thus, in some embodiments, treatment of such pain involves stimulation of at least a portion of the gray ramus communicans nerves.

The intervertebral discs are soft tissue structures with an outer annulus of fibrous tissue and an inner core (or nucleus) of softer tissue referred to as the nucleus pulposus situated between the vertebra. Innervation of the intervertebral discs stem both from sympathetic afferents as well as somatic afferent nerves. The somatic nerves course through a normal pathways through the sensory nervous system (through the DRG) and on to the central nervous system. The sympathetic afferent nerves innervating the intervertebral discs can course through the sinuvertebral nerve that enters the central nervous system via the L2 dorsal root ganglion (DRG). Thus, the L2 dorsal root ganglion is a key target in the treatment of discogenic pain. When disruptions to the disc occur resulting in chronic pain, sympathetic nerve sprouting is noted within the disc demonstrating an enhanced innervation pattern that contributes to the chronic pain condition. By targeting the L2 DRG an enhanced ability to treat discogenic low back pain may be realized. Discogenic pain from disc disruption can be mechanically based with a component of neuropathic pain from the rich sympathetic afferent growth observed. Treatment of the L2 ganglion would potentially be able to treat both the mechanically oriented pain condition as well as the neuropathic component due to the innervation patterns.

### Treatment of Other Portions of the Peripheral Nervous System

As mentioned, in some embodiments the systems and devices are used to stimulate other portions of the peripheral nervous system. The peripheral nervous system is commonly subdivided into two subsystems, the somatic nervous system and the autonomic nervous system. In some embodiments, the systems and devices of the present invention are used to selectively stimulate nerves of the autonomic nervous system. Examples of such target nerves are as follows:

### Nerves of the Autonomic Nervous System

1) Head/cranial
a) Sympathetic
   Ciliary ganglion: roots (Sensory, Sympathetic), Short ciliary
   Ciliary ganglion: roots (Sensory, Parasympathetic), Short ciliary
   Pterygopalatine ganglion: deep petrosal, nerve of pterygoid canal
b) Parasympathetic
   branches of distribution: greater palatine (inferior posterior nasal branches of greater palatine nerve), lesser palatine, nasopalatine (medial superior posterior nasal branches), pharyngeal
   Submandibular ganglion
   Otic ganglion

2) Neck/cervical
a) Sympathetic
   Paravertebral ganglia: Cervical ganglia (Superior, Middle, Inferior), Stellate ganglion
   Prevertebral plexus: Cavernous plexus, Internal carotid

3) Chest/thorax
a) Sympathetic
   Paravertebral ganglia: Thoracic ganglia
   Prevertebral plexus: Cardiac plexus, Esophageal plexus, Pulmonary plexus, Thoracic aortic plexus
   Splanchnic nerves: cardiopulmonary, thoracic
   Cardiac nerves: Superior, Middle, Inferior

4) Abdomen/Lumbar
a) Sympathetic
   Paravertebral ganglia: Lumbar ganglia
   Prevertebral ganglia: Celiac ganglia (Aorticorenal), Superior mesenteric ganglion, Inferior mesenteric ganglion
   Prevertebral plexus: Celiac plexus, (Hepatic, Splenic, Pancreatic), aorticorenal (Abdominal aortic plexus, Renal/Suprarenal), Superior mesenteric (Gastric), Inferior mesenteric (Spermatic, Ovarian), Superior hypogastric (hypogastric nerve, Superior rectal), Inferior hypogastric (Vesical, Prostatic/Cavernous nerves of penis, Uterovaginal, Middle rectal)
   Splanchnic nerves: Lumbar splanchnic nerves
b) Enteric
   Meissner's plexus
   Auerbach's plexus

5) Pelvis/sacral
a) Sympathetic
   Paravertebral ganglia: Sacral ganglia, Ganglion impar
   Splanchnic nerves: Sacral splanchnic nerves
b) Parasympathetic
   Splanchnic nerves: Pelvic splanchnic nerves

In some embodiments, the systems and devices of the present invention are used to selectively stimulate cranial nerves (CN), such as CN I—Olfactory, CN II—Optic, CN III—Oculomotor, CN IV—Trochlear, CN V—Trigeminal, CN VI—Abducens, CN VII—Facial, CN VIII—Vestibulocochlear, CN IX—Glossopharyngeal, CN X—Vagus, CN XI—Accessory, and CN XII—Hypoglossal. In particular embodiments, the systems and devices of the present invention are used to selectively stimulate portions of the trigeminal nerve. Examples of such target portions of the trigeminal nerve are as follows

### Cranial Nerve: Trigeminal Nerve

A) Ophthalmic
a) frontal: supratrochlear, supraorbital (lateral branch, medial branch)
b) nasociliary: long ciliary, infratrochlear, posterior ethmoidal, anterior ethmoidal (external nasal, internal nasal), sensory root of ciliary ganglion (ciliary ganglion)
c) lacrimal

B) Maxillary
a) in meninges: middle meningeal
b) in pterygopalatine fossa: zygomatic (zygomaticotemporal, zygomaticofacial), pterygopalatine (pterygopalatine ganglion), posterior superior alveolar
c) in infraorbital canal: infraorbital nerve: superior alveolar (middle, anterior), internal nasal branches
d) on face: inferior palpebral, external nasal, superior labial (infraorbital plexus)

C) Mandibular
a) in meninges: meningeal
b) anterior: to muscles of mastication (medial pterygoid/to tensor veli palatini, lateral pterygoid, masseteric, deep temporal), buccal
c) posterior: auriculotemporal (otic ganglion), lingual (submandibular ganglion), inferior alveolar (mylohyoid, mental)

The trigeminal nerve is the largest of the cranial nerves. The trigeminal nerve has three major branches: the ophthalmic nerve, the maxillary nerve, and the mandibular nerve. The ophthalmic and maxillary nerves are purely sensory. The mandibular nerve has both sensory and motor functions. The three branches converge on the trigeminal ganglion (also called the semilunar ganglion or gasserian ganglion), that is located within Meckel's cave, and contains the cell bodies of incoming sensory nerve fibers. The trigeminal ganglion contains the cell bodies of sensory fibers incoming from the rest of the body. From the trigeminal ganglion, a single large sensory root enters the brainstem at the level of the pons. Immediately adjacent to the sensory root, a smaller motor root emerges from the pons at the same level. Motor fibers pass through the trigeminal ganglion on their way to peripheral muscles, but their cell bodies are located in the nucleus of the fifth nerve, deep within the pons.

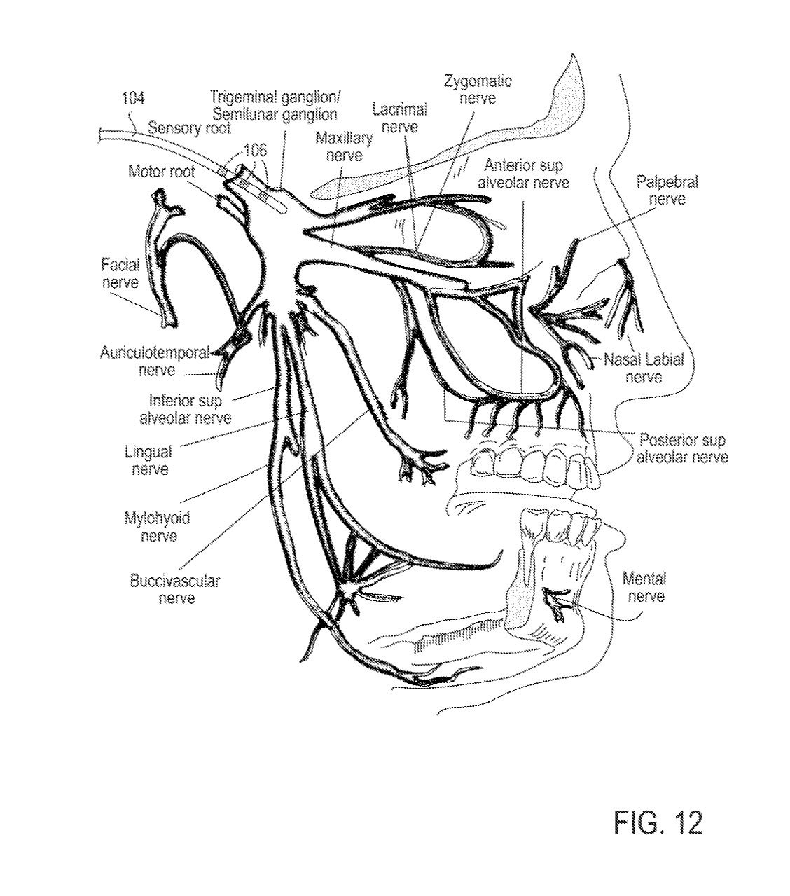

FIG. 12 illustrates an embodiment of a lead 104 of the present invention positioned so as to selectively stimulate the trigeminal ganglion. Selective stimulation of the trigeminal ganglion is achieved with the choice of the size of the electrode(s), the shape of the electrode(s), the position of the electrode(s), the stimulation signal, pattern or algorithm, or any combination of these. Such selective stimulation stimulates the targeted neural tissue while excluding untargeted tissue, such as surrounding or nearby tissue. In some embodiments, the stimulation energy is delivered to the targeted neural tissue so that the energy dissipates or attenuates beyond the targeted tissue or region to a level insufficient to stimulate modulate or influence such untargeted tissue. In particular, selective stimulation of the trigeminal ganglion excludes stimulation of the nearby motor root wherein the stimulation signal has an energy below an energy threshold for stimulating the motor root while the lead is so positioned. In some embodiments, the methods and devices to achieve such selective stimulation are provided in U.S. patent application Ser. No. 12/607,009, entitled “Selective Stimulation Systems and Signal Parameters for Medical Conditions” and U.S. patent application Ser. No. 12/687,737, entitled “Stimulation Leads, Delivery Systems and Methods of Use”, both incorporated herein by reference for all purposes. In other embodiments, methods and devices to selectively stimulate the trigeminal ganglion are specific to the anatomy of the trigeminal ganglion and/or the condition treated. In particular, the stimulation signal parameters may include a pulse width, frequency, amplitude or a combination of these which selectively stimulates the trigeminal ganglion or anatomies within the trigeminal ganglion while excluding or minimizing the stimulation of surrounding or other anatomies. In some embodiments, the devices include specialized delivery devices for delivering and positioning the lead near the trigeminal ganglion. The lead may also have particular features, such as small diameter and high flexibility which enable desirable placement and reduced migration in the area of the trigeminal ganglion.

The ophthalmic, maxillary and mandibular branches leave the skull through three separate foramina: the superior orbital fissure, the foramen rotundum and the foramen ovale, respectively. The ophthalmic nerve carries sensory information from the scalp and forehead, the upper eyelid, the conjunctiva and cornea of the eye, the nose (including the tip of the nose, except alae nasi), the nasal mucosa, the frontal sinuses, and parts of the meninges (the dura and blood vessels). The maxillary nerve carries sensory information from the lower eyelid and cheek, the nares and upper lip, the upper teeth and gums, the nasal mucosa, the palate and roof of the pharynx, the maxillary, ethmoid and sphenoid sinuses, and parts of the meninges. The mandibular nerve carries sensory information from the lower lip, the lower teeth and gums, the chin and jaw (except the angle of the jaw, which is supplied by C2-C3), parts of the external ear, and parts of the meninges. The mandibular nerve carries touch/position and pain/temperature sensation from the mouth. It does not carry taste sensation (chorda tympani is responsible for taste), but one of its branches, the lingual nerve, carries multiple types of nerve fibers that do not originate in the mandibular nerve.

The lead 104 may be advanced beyond the trigeminal ganglion, through the various foramina, including the superior orbital fissure, the foramen rotundum and the foramen ovale, so as to position the electrodes 106 along a desired target nerve within the head or neck. In order to achieve such positioning, the lead 104 is appropriately sized and configured to maneuver through the facial anatomy. Consequently, the lead 104 is significantly smaller and more easily maneuverable than conventional spinal cord stimulator leads. In some embodiments, the leads and delivery systems used to reach such target sites within the anatomy are provided in U.S. patent application Ser. No. 12/687,737, entitled “Stimulation Leads, Delivery Systems and Methods of Use”, incorporated herein by reference for all purposes.

It may be appreciated that the lead 104 may be advanced to the trigeminal ganglion or other targets within the head, neck and face via atraumatic epidural advancement along the spinal cord S in an antegrade direction. However, it may also be appreciated that these targets may be reached by other access routes, including direct access through the skin to the target site. In some embodiments, the target nerve is accessed with the use of a needle under guided visualization, such as guided ultrasound. The lead 104 is advanced through the needle so as to position at least one of the electrodes 106 near or on the portion of the nerve to receive the stimulation. It may be appreciated that in some embodiments, at least one of the electrodes 106 is positioned within the nerve. In some embodiments, the lead 104 is so positioned with the use of an introducer which has a stiffness suitable for advancement through the tissue near the target nerve anatomy. Likewise, in some embodiments, the lead is tunneled through the tissue with the use of one or more dilators. It may also be appreciated that in some embodiments the lead 104 is implanted via conventional surgical methods, or using other endoscopic or laparoscopic techniques.

The systems, devices and methods of the present invention may be used to treat a variety of disorders related to the head, face and neck, such as pain originating in or manifesting in any of these regions. Such pain may result from one or more medical conditions including migraines, headaches, post-traumatic neck pain, post-herpetic neuralgia involving the head, face or neck, myaliga of neck muscles, temporomandibular joint disorder, intracranial hypertension, arthritis, otalgia due to middle ear lesion, maxillary neuralgia, laryngeal pain, and sphenopalatine ganglion neuralgia to name a few.

In classical anatomy, most sensory information from the face is carried by the trigeminal nerve, but sensation from certain parts of the mouth, certain parts of the ear and certain parts of the meninges is carried by “general somatic afferent” fibers in cranial nerves VII (the facial nerve), IX (the glossopharyngeal nerve) and X (the vagus nerve). Without exception, however, all sensory fibers from these nerves terminate in the trigeminal nucleus. On entering the brainstem, sensory fibers from V, VII, IX, and X are sorted out and sent to the trigeminal nucleus, which thus contains a complete sensory map of the face and mouth. The spinal counterparts of the trigeminal nucleus (cells in the dorsal horn and dorsal column nuclei of the spinal cord) contain a complete sensory map of the rest of the body. The trigeminal nucleus extends throughout the entire brainstem, from the midbrain to the medulla, and continues into the cervical cord, where it merges with the dorsal horn cells of the spinal cord. The nucleus is divided anatomically into three parts, visible in microscopic sections of the brainstem. From caudal to rostral (i.e., going up from the medulla to the midbrain) they are the spinal trigeminal nucleus, the main trigeminal nucleus, and the mesencephalic trigeminal nucleus.

The three parts of the trigeminal nucleus receive different types of sensory information. The spinal trigeminal nucleus receives pain/temperature fibers. The main trigeminal nucleus receives touch/position fibers. The mesencephalic nucleus receives proprioceptor and mechanoreceptor fibers from the jaws and teeth.

The spinal trigeminal nucleus represents pain/temperature sensation from the face. Pain/temperature fibers from peripheral nociceptors are carried in cranial nerves V, VII, IX, and X. On entering the brainstem, sensory fibers are grouped together and sent to the spinal trigeminal nucleus. This bundle of incoming fibers can be identified in cross sections of the pons and medulla as the spinal tract of the trigeminal nucleus, which parallels the spinal trigeminal nucleus itself.

The spinal trigeminal nucleus contains a pain/temperature sensory map of the face and mouth. From the spinal trigeminal nucleus, secondary fibers cross the midline and ascend in the trigeminal lemniscus to the contralateral thalamus. The trigeminal lemniscus runs parallel to the medial lemniscus, which carries pain/temperature information to the thalamus from the rest of the body. Pain/temperature fibers are sent to multiple thalamic nuclei. The central processing of pain/temperature information is markedly different from the central processing of touch/position information.

The main trigeminal nucleus represents touch/position sensation from the face. It is located in the pons, close to the entry site of the fifth nerve. Fibers carrying carry touch/position information from the face and mouth (via cranial nerves V, VII, IX, and X) are sent to the main trigeminal nucleus when they enter the brainstem. The main trigeminal nucleus contains a touch/position sensory map of the face and mouth, just as the spinal trigeminal nucleus contains a complete pain/temperature map.

From the main trigeminal nucleus, secondary fibers cross the midline and ascend in the trigeminal lemniscus to the contralateral thalamus. The trigeminal lemniscus runs parallel to the medial leminscus, which carries touch/position information from the rest of the body to the thalamus. Some sensory information from the teeth and jaws is sent from the main trigeminal nucleus to the ipsilateral thalamus, via the small dorsal trigeminal tract. Thus touch/position information from the teeth and jaws is represented bilaterally in the thalamus (and hence in the cortex). The reason for this special processing is discussed below.

The mesencephalic trigeminal nucleus is not really a “nucleus.” Rather, it is a sensory ganglion that happens to be embedded in the brainstem. The mesencephalic “nucleus” is the sole exception to the general rule that sensory information passes through peripheral sensory ganglia before entering the central nervous system. Only certain types of sensory fibers have cell bodies in the mesencephalic nucleus: proprioceptor fibers from the jaw and mechanoreceptor fibers from the teeth. Some of these incoming fibers go to the motor nucleus of V, thus entirely bypassing the pathways for conscious perception. The jaw jerk reflex is an example. Tapping the jaw elicits a reflex closure of the jaw, in exactly the same way that tapping the knee elicits a reflex kick of the lower leg. Other incoming fibers from the teeth and jaws go to the main nucleus of V. As noted above, this information is projected bilaterally to the thalamus. It is available for conscious perception.

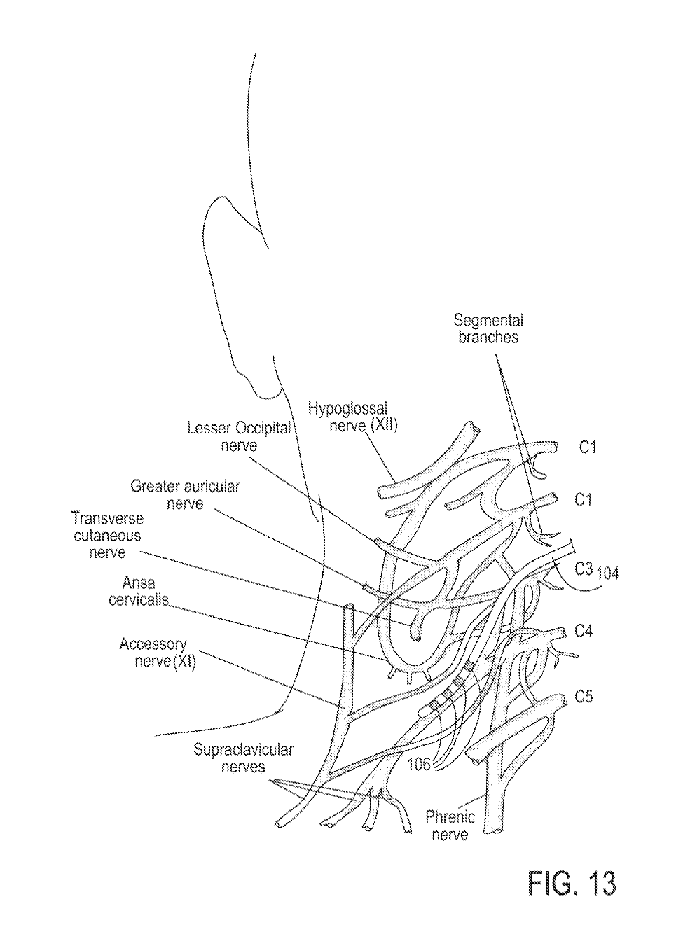

In some embodiments, the systems and devices of the present invention are used to selectively stimulate portions of the cervical plexus. Referring to FIG. 13, the cervical plexus is a plexus of the ventral rami of the first four cervical spinal nerves which are located from C1 to C5 cervical segment in the neck. They are located laterally to the transverse processes between prevertebral muscles from the medial side and vertebral (m. scalenus, m. levator scapulae, m. splenius cervicis) from lateral side. There is anastomosis with accessory nerve, hypoglossal nerve and sympathetic trunk. The cervical plexus is located in the neck, deep to sternocleidomastoid. Nerves formed from the cervical plexus innervate the back of the head, as well as some neck muscles. The branches of the cervical plexus emerge from the posterior triangle at the nerve point, a point which lies midway on the posterior border of the sternocleidomastoid. In one embodiment, the lead 104 is advanced from cervical segment C3 and positioned within the cervical plexus, as shown in FIG. 13. Here, the lead 104 is positioned along a nerve leading to the supraclavicular nerves. It may be appreciated that the lead 104 may similarly be positioned along any of the nerves of the cervical plexus. Referring again to FIG. 13, the lead 104 is positioned so as to selectively stimulate a portion of the cervical plexus. Selective stimulation is achieved with the choice of the size of the electrode(s), the shape of the electrode(s), the position of the electrode(s), the stimulation signal, pattern or algorithm, or any combination of these. Such selective stimulation stimulates the targeted neural tissue while excluding untargeted tissue, such as surrounding or nearby tissue. In some embodiments, the stimulation energy is delivered to the targeted neural tissue so that the energy dissipates or attenuates beyond the targeted tissue or region to a level insufficient to stimulate modulate or influence such untargeted tissue. In some embodiments, the methods and devices to achieve such selective stimulation are provided in U.S. patent application Ser. No. 12/607,009, entitled “Selective Stimulation Systems and Signal Parameters for Medical Conditions”, incorporated herein by reference for all purposes. In other embodiments, methods and devices to selectively stimulate the cervical plexus are specific to the anatomy of the nerves of the cervical plexus and/or the condition treated. In particular, the stimulation signal parameters may include a pulse width, frequency, amplitude or a combination of these which selectively stimulates the nerves of the cervical plexus while excluding or minimizing the stimulation of surrounding or other anatomies.

The lead 104 may be advanced along portions of the cervical plexus so as to desirably position the electrodes 106 along the target nerve. In order to achieve such positioning, the lead 104 is appropriately sized and configured to maneuver through the anatomy of the neck. Consequently, the lead 104 is significantly smaller and more easily maneuverable than conventional spinal cord stimulator leads. In some embodiments, the leads and delivery systems used to reach such target sites within the anatomy are provided in U.S. patent application Ser. No. 12/687,737, entitled “Stimulation Leads, Delivery Systems and Methods of Use”, incorporated herein by reference for all purposes. In some embodiments, the devices include specialized delivery devices for delivering and positioning the lead near the nerves of the cervical plexus. The lead may also have particular features, such as small diameter and high flexibility which enable desirable placement and reduced migration in the area of the cervical plexus.

It may be appreciated that the lead 104 may be advanced to portions of the cervical plexus or other targets within or beyond the cervical plexus via atraumatic epidural advancement along the spinal cord S and exiting through a cervical foramen. However, it may also be appreciated that these targets may be reached by other access routes, including direct access through the skin to the target site. In some embodiments, the target nerve is accessed with the use of a needle under guided visualization, such as guided ultrasound. The lead 104 is advanced through the needle so as to position at least one of the electrodes 106 near or on the portion of the nerve to receive the stimulation. It may be appreciated that in some embodiments, at least one of the electrodes 106 is positioned within the nerve. In some embodiments, the lead 104 is so positioned with the use of an introducer which has a stiffness suitable for advancement through the tissue near the target nerve anatomy. Likewise, in some embodiments, the lead is tunneled through the tissue with the use of one or more dilators. It may also be appreciated that in some embodiments the lead 104 is implanted via conventional surgical methods, or using other endoscopic or laparoscopic techniques.

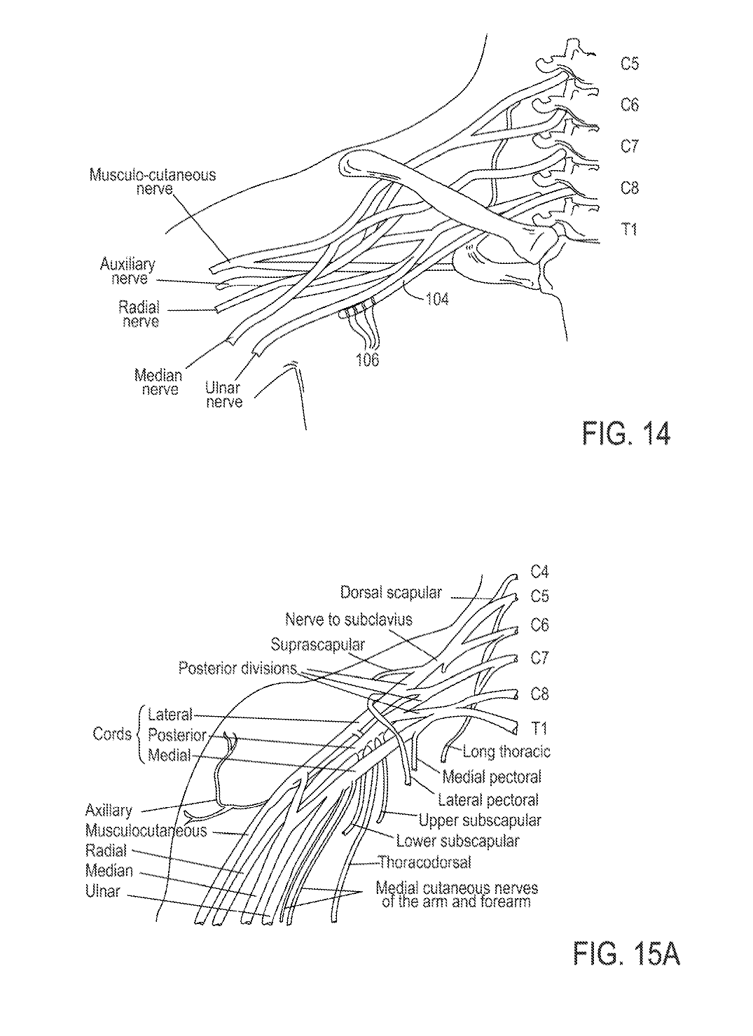

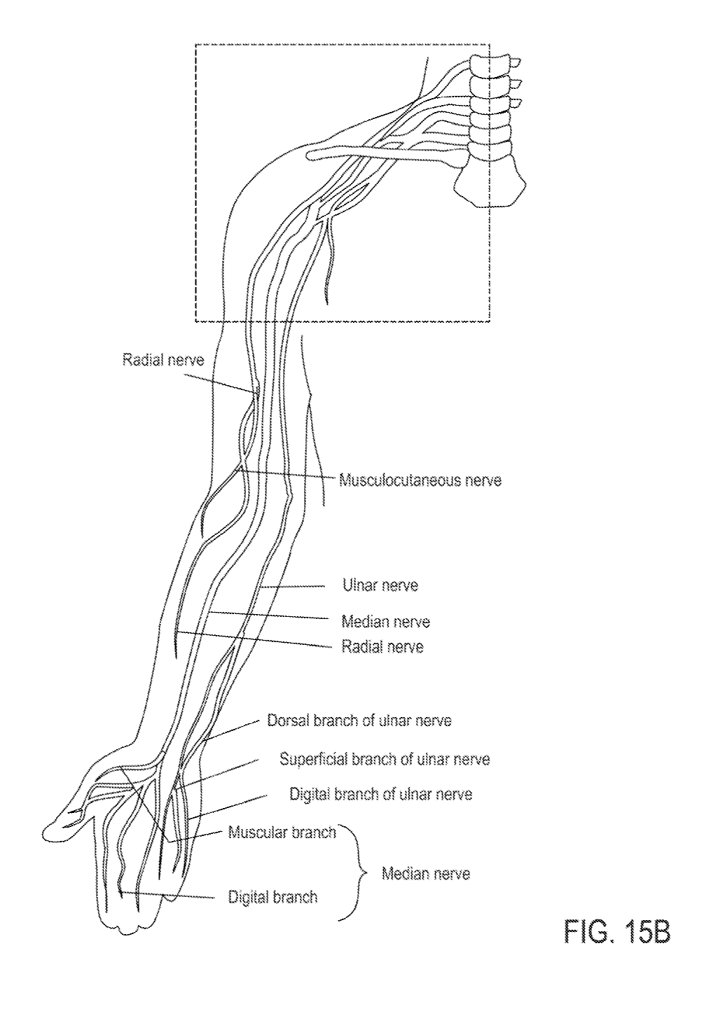

In some embodiments, the systems and devices of the present invention are used to selectively stimulate portions of the brachial plexus. Referring to FIG. 14, the brachial plexus is an arrangement of nerve fibers, running from the spine, formed by the ventral rami of the lower four cervical and first thoracic nerve roots (C5-T1). It proceeds through the neck, the axilla (armpit region), and into the arm. FIG. 15A provides another view of the brachial plexus anatomy residing in the shoulder and FIG. 15B illustrates the nerves extending into the arm and hand. In one embodiment, the lead 104 is advanced from cervical segment C8 and positioned within the brachial plexus, as shown in FIG. 14. Here, the lead 104 is positioned along a nerve leading to the ulnar nerve. It may be appreciated that the lead 104 may similarly be positioned along any of the nerves of the brachial plexus. Referring again to FIG. 14, the lead 104 is positioned so as to selectively stimulate a portion of the brachial plexus. Selective stimulation is achieved with the choice of the size of the electrode(s), the shape of the electrode(s), the position of the electrode(s), the stimulation signal, pattern or algorithm, or any combination of these. Such selective stimulation stimulates the targeted neural tissue while excluding untargeted tissue, such as surrounding or nearby tissue. In some embodiments, the stimulation energy is delivered to the targeted neural tissue so that the energy dissipates or attenuates beyond the targeted tissue or region to a level insufficient to stimulate modulate or influence such untargeted tissue, such as nerves leading to other regions of the arm and hand. In some embodiments, the methods and devices to achieve such selective stimulation are provided in U.S. patent application Ser. No. 12/607,009, entitled “Selective Stimulation Systems and Signal Parameters for Medical Conditions”, incorporated herein by reference for all purposes. In other embodiments, methods and devices to selectively stimulate the brachial plexus are specific to the anatomy of the nerves of the brachial plexus and/or the condition treated. In particular, the stimulation signal parameters may include a pulse width, frequency, amplitude or a combination of these which selectively stimulates the nerves of the brachial plexus while excluding or minimizing the stimulation of surrounding or other anatomies.

The lead 104 may be advanced along portions of the brachial plexus so as to desirably position the electrodes 106 along the target nerve. In order to achieve such positioning, the lead 104 is appropriately sized and configured to maneuver through the local anatomy. Consequently, the lead 104 is significantly smaller and more easily maneuverable than conventional spinal cord stimulator leads. In some embodiments, the leads and delivery systems used to reach such target sites within the anatomy are provided in U.S. patent application Ser. No. 12/687,737, entitled “Stimulation Leads, Delivery Systems and Methods of Use”, incorporated herein by reference for all purposes. In some embodiments, the devices include specialized delivery devices for delivering and positioning the lead near the nerves of the brachial plexus. The lead may also have particular features, such as small diameter and high flexibility which enable desirable placement and reduced migration in the area of the brachial plexus.

It may be appreciated that the lead 104 may be advanced to portions of the brachial plexus or other targets within or beyond the brachial plexus via atraumatic epidural advancement along the spinal cord S and exiting through a cervical or thoracic foramen. However, it may also be appreciated that these targets may be reached by other access routes, including direct access through the skin to the target site, such as in directly into the arm or hand. In some embodiments, the target nerve is accessed with the use of a needle under guided visualization, such as guided ultrasound. The lead 104 is advanced through the needle so as to position at least one of the electrodes 106 near or on the portion of the nerve to receive the stimulation. It may be appreciated that in some embodiments, at least one of the electrodes 106 is positioned within the nerve. In some embodiments, the lead 104 is so positioned with the use of an introducer which has a stiffness suitable for advancement through the tissue near the target nerve anatomy. Likewise, in some embodiments, the lead is tunneled through the tissue with the use of one or more dilators. It may also be appreciated that in some embodiments the lead 104 is implanted via conventional surgical methods, or using other endoscopic or laparoscopic techniques.

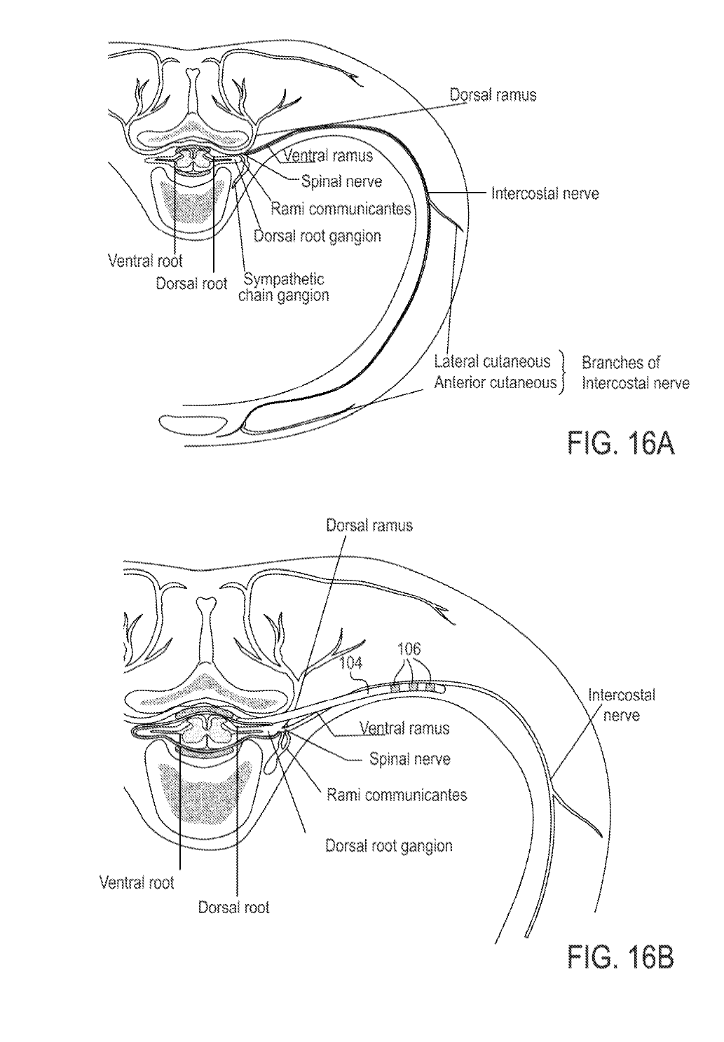

In some embodiments, the systems and devices of the present invention are used to selectively stimulate portions of the peripheral nervous system throughout the thoracic region. FIG. 16A provides a cross-sectional view of the thoracic cavity and associated spinal anatomy. As shown, the peripheral nervous system extends to the sympathetic chain ganglions, the dorsal ramus and the ventral ramus. The ventral ramus extends to the intercostal nerves. The intercostal nerves are the anterior divisions (rami anteriores; ventral divisions) of the thoracic spinal nerves from T1 to T11. Each nerve is connected with the adjoining ganglion of the sympathetic trunk by a gray and a white ramus communicans. The intercostal nerves are distributed chiefly to the thoracic pleura and abdominal peritoneum and differ from the anterior divisions of the other spinal nerves in that each pursues an independent course without plexus formation. The first two nerves supply fibers to the upper limb in addition to their thoracic branches; the next four are limited in their distribution to the parietes of the thorax; the lower five supply the parietes of the thorax and abdomen. The 7th intercostal nerve terminates at the xyphoid process, at the lower end of the sternum. The 10th intercostal nerve terminates at the umbilicus. The twelfth (subcostal) thoracic is distributed to the abdominal wall and groin.

In one embodiment, illustrated in FIG. 16B, the lead 104 is advanced epidurally along the spinal cord S, through a sharp curve toward a DRG, and through a foramen wherein the distal end of the lead 104 is positioned along an intercostal nerve. It may be appreciated that the lead 104 may similarly be positioned along any of the nerves of the thoracic region. Referring again to FIG. 16B, the lead 104 is positioned so as to selectively stimulate a portion of the intercostal nerve. Selective stimulation is achieved with the choice of the size of the electrode(s), the shape of the electrode(s), the position of the electrode(s), the stimulation signal, pattern or algorithm, or any combination of these. Such selective stimulation stimulates the targeted neural tissue while excluding untargeted tissue, such as surrounding or nearby tissue. In some embodiments, the stimulation energy is delivered to the targeted neural tissue so that the energy dissipates or attenuates beyond the targeted tissue or region to a level insufficient to stimulate modulate or influence such untargeted tissue, such as nerves leading to other regions of the thoracic cavity or points beyond. In some embodiments, the methods and devices to achieve such selective stimulation are provided in U.S. patent application Ser. No. 12/607,009, entitled “Selective Stimulation Systems and Signal Parameters for Medical Conditions”, incorporated herein by reference for all purposes. In other embodiments, methods and devices to selectively stimulate the intercostal nerve are specific to the anatomy of the nerves of the thoracic region and/or the condition treated. In particular, the stimulation signal parameters may include a pulse width, frequency, amplitude or a combination of these which selectively stimulates the intercostal nerve while excluding or minimizing the stimulation of surrounding or other anatomies.

The lead 104 may be advanced along portions of the intercostal nerves so as to desirably position the electrodes 106 along the target nerve. In order to achieve such positioning, the lead 104 is appropriately sized and configured to maneuver through the epidural space, through a foramen and within the local anatomy. Consequently, the lead 104 is significantly smaller and more easily maneuverable than conventional spinal cord stimulator leads. In some embodiments, the leads and delivery systems used to reach such target sites within the anatomy are provided in U.S. patent application Ser. No. 12/687,737, entitled “Stimulation Leads, Delivery Systems and Methods of Use”, incorporated herein by reference for all purposes. In some embodiments, the devices include specialized delivery devices for delivering and positioning the lead near the nerves of the thoracic region, particularly the intercostal nerve. The lead may also have particular features, such as small diameter and high flexibility which enable desirable placement and reduced migration in the area of the interocostal nerve.

It may be appreciated that the lead 104 may be advanced to portions of the thoracic region or beyond by other access routes, including direct access through the skin to the target site, such as in directly into the torso. In some embodiments, the target nerve is accessed with the use of a needle under guided visualization, such as guided ultrasound. The lead 104 is advanced through the needle so as to position at least one of the electrodes 106 near or on the portion of the nerve to receive the stimulation. It may be appreciated that in some embodiments, at least one of the electrodes 106 is positioned within the nerve. In some embodiments, the lead 104 is so positioned with the use of an introducer which has a stiffness suitable for advancement through the tissue near the target nerve anatomy. Likewise, in some embodiments, the lead is tunneled through the tissue with the use of one or more dilators. It may also be appreciated that in some embodiments the lead 104 is implanted via conventional surgical methods, or using other endoscopic or laparoscopic techniques.

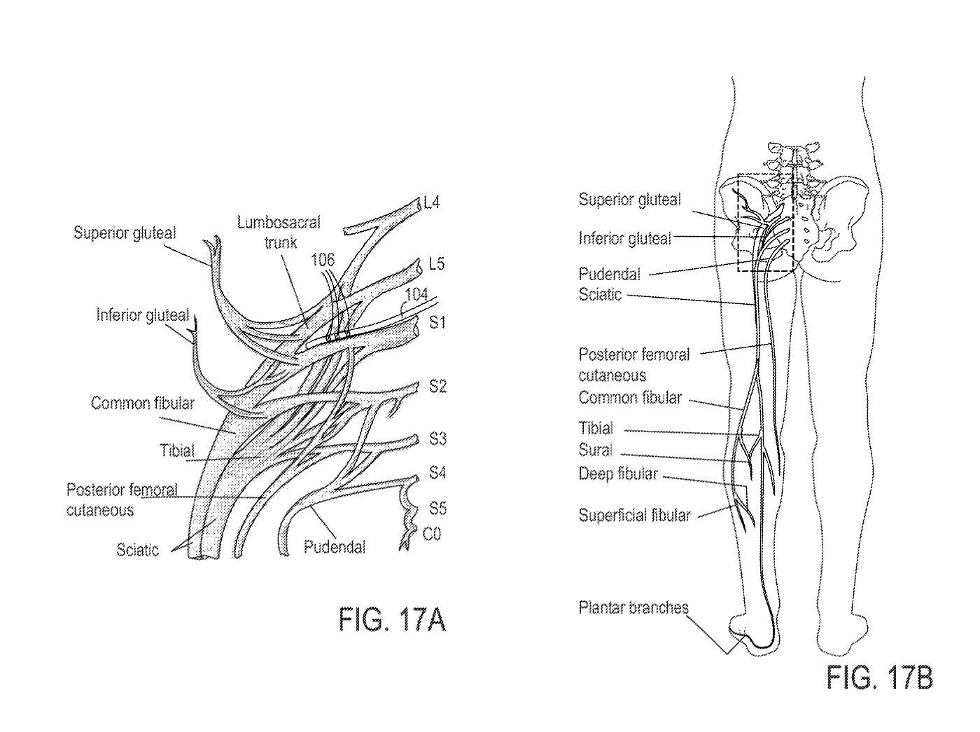

In some embodiments, the systems and devices of the present invention are used to selectively stimulate portions of the sacral plexus. Referring to FIGS. 17A-17B, the sacral plexus is a nerve plexus which provides motor and sensory nerves for the posterior thigh, most of the lower leg, the entire foot, and part of the pelvis. It is part of the lumbosacral plexus and emerges from the sacral vertebrae (S2-S4). The sacral plexus is formed by the lumbosacral trunk, the anterior division of the first sacral nerve, and portions of the anterior divisions of the second and third sacral nerves. The nerves forming the sacral plexus converge toward the lower part of the greater sciatic foramen, and unite to form a flattened band, from the anterior and posterior surfaces of which several branches arise. The band itself is continued as the sciatic nerve, which splits on the back of the thigh into the tibial nerve and common fibular nerve; these two nerves sometimes arise separately from the plexus, and in all cases their independence can be shown by dissection. Often, the sacral plexus and the lumbar plexus are considered to be one large nerve plexus, the lumbosacral plexus. The lumbosacral trunk connects the two plexuses.

In one embodiment, the lead 104 is advanced from sacral segment S1 and positioned within the sacral plexus, as shown in FIG. 17A. Here, the lead 104 is positioned along a nerve leading to the common fibular nerve. It may be appreciated that the lead 104 may similarly be positioned along any of the nerves of the sacral plexus. Referring again to FIG. 17A, the lead 104 is positioned so as to selectively stimulate a portion of the sacral plexus. Selective stimulation is achieved with the choice of the size of the electrode(s), the shape of the electrode(s), the position of the electrode(s), the stimulation signal, pattern or algorithm, or any combination of these. Such selective stimulation stimulates the targeted neural tissue while excluding untargeted tissue, such as surrounding or nearby tissue. In some embodiments, the stimulation energy is delivered to the targeted neural tissue so that the energy dissipates or attenuates beyond the targeted tissue or region to a level insufficient to stimulate modulate or influence such untargeted tissue, such as nerves leading to other regions of the leg or foot. In some embodiments, the methods and devices to achieve such selective stimulation are provided in U.S. patent application Ser. No. 12/607,009, entitled “Selective Stimulation Systems and Signal Parameters for Medical Conditions”, incorporated herein by reference for all purposes. In other embodiments, methods and devices to selectively stimulate any of the nerves of the sacral plexus are specific to the anatomy of the nerves of the sacral plexus and/or the condition treated. In particular, the stimulation signal parameters may include a pulse width, frequency, amplitude or a combination of these which selectively stimulates a nerve of the sacral plexus or any other nerve in the region while excluding or minimizing the stimulation of surrounding or other anatomies.

The lead 104 may be advanced along portions of the sacral plexus so as to desirably position the electrodes 106 along the target nerve. In order to achieve such positioning, the lead 104 is appropriately sized and configured to maneuver through the local anatomy. Consequently, the lead 104 is significantly smaller and more easily maneuverable than conventional spinal cord stimulator leads. In some embodiments, the leads and delivery systems used to reach such target sites within the anatomy are provided in U.S. patent application Ser. No. 12/687,737, entitled “Stimulation Leads, Delivery Systems and Methods of Use”, incorporated herein by reference for all purposes. In some embodiments, the devices include specialized delivery devices for delivering and positioning the lead near the nerves of the sacral plexus. The lead may also have particular features, such as small diameter and high flexibility which enable desirable placement and reduced migration in the area of the sacral plexus.

It may be appreciated that the lead 104 may be advanced to portions of the sacral plexus or other targets within or beyond the sacral plexus via atraumatic epidural advancement along the spinal cord S and exiting through a sacral foramen. However, it may also be appreciated that these targets may be reached by other access routes, including direct access through the skin to the target site, such as in directly into the leg or foot. In some embodiments, the target nerve is accessed with the use of a needle under guided visualization, such as guided ultrasound. The lead 104 is advanced through the needle so as to position at least one of the electrodes 106 near or on the portion of the nerve to receive the stimulation. It may be appreciated that in some embodiments, at least one of the electrodes 106 is positioned within the nerve. In some embodiments, the lead 104 is so positioned with the use of an introducer which has a stiffness suitable for advancement through the tissue near the target nerve anatomy. Likewise, in some embodiments, the lead is tunneled through the tissue with the use of one or more dilators. It may also be appreciated that in some embodiments the lead 104 is implanted via conventional surgical methods, or using other endoscopic or laparoscopic techniques.

### Approaching Target Anatomies

As mentioned, any of the target anatomies can be accessed via an epidural approach, wherein the lead is advanced along the spinal cord S, curves laterally away from the midline, is advanced along a nerve root and (for target anatomies beyond the dorsal root ganglion) exits the epidural space through a foramen. In some embodiments, the epidural approach is an antegrade approach. It may be appreciated that, alternatively, other approaches may be used, such as a retrograde approach or a contralateral approach.

By approaching target anatomies in the peripheral nervous system via an epidural approach, multiple points of mechanical stability are provided to the lead. To begin, the epidural space acts as a lead stabilization pathway. As the spine is a stiff, stable structure, the lead is protected from major movements that would place strain on the lead and tend to propagate migration. In addition, at the point of exiting the foramen, the lead is also stabilized by the boney opening and soft tissue within the foramen. And further, anchoring techniques near the spine provide a more stable grounding or anchoring of the lead to the anatomy (the spine and adjacent fascia) compared to the soft tissues in the periphery.

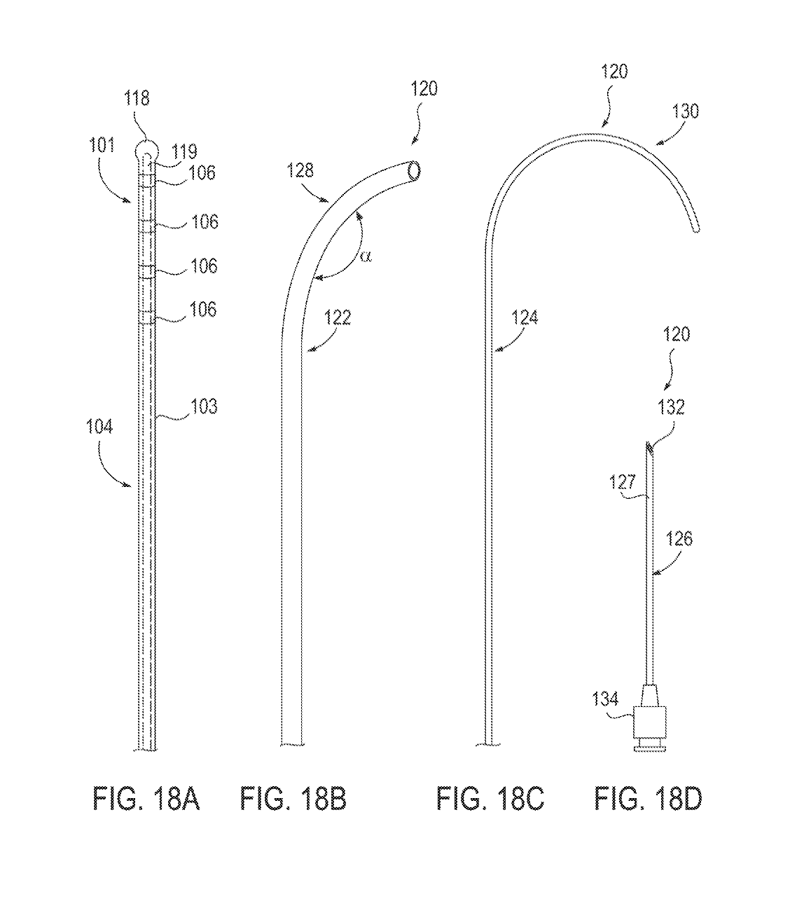

In some embodiments, the lead is delivered to the dorsal root ganglion and/or targets beyond the foramen in the peripheral nervous system with the use of a delivery system. FIGS. 18A-18B illustrate an embodiment of a lead 104 (FIG. 8A) and delivery system 120, including a sheath 122 (FIG. 8B), stylet 124 (FIG. 8C) and introducing needle 126 (FIG. 8D), for such purposes. In this embodiment, the lead 104 comprises a shaft 103 having a distal end 101 and four electrodes 106 disposed thereon. It may be appreciated that any number of electrodes 106 may be present, including one, two, three, four, five, six, seven, eight or more. In this embodiment, the distal end 101 has a closed-end distal tip 118. The distal tip 118 may have a variety of shapes including a rounded shape, such as a ball shape (shown) or tear drop shape, and a cone shape, to name a few. These shapes provide an atraumatic tip for the lead 104 as well as serving other purposes. The lead 104 also includes a stylet lumen 119 which extends toward the closed-end distal tip 118.

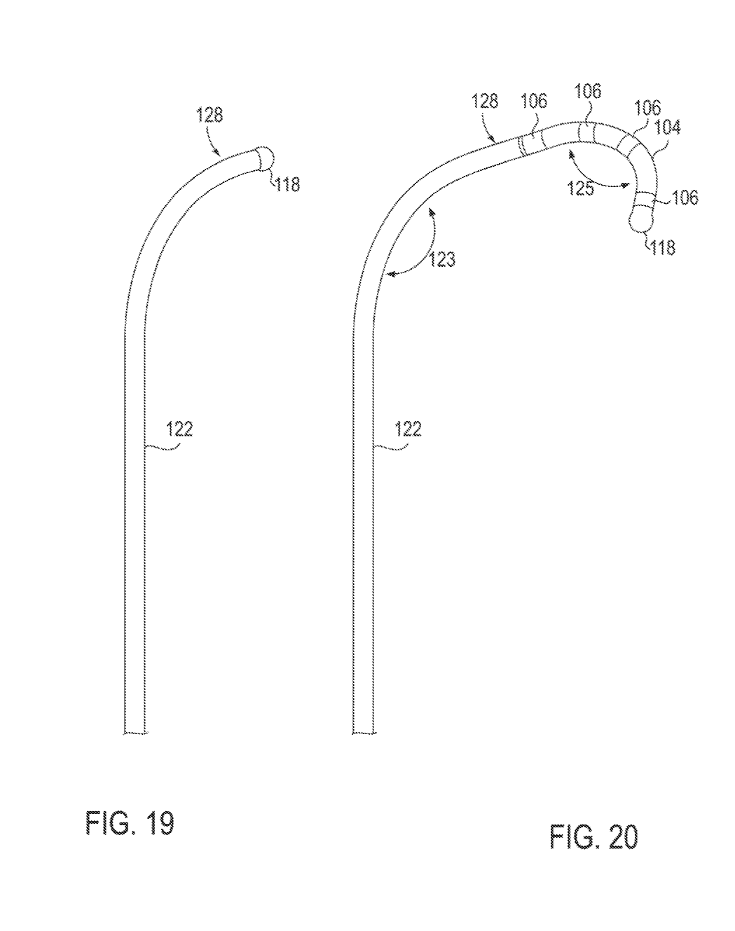

FIG. 18B illustrates an example embodiment of a sheath 122. The sheath 122 has a distal end 128 which is pre-curved to have an angle α, wherein the angle α is in the range of approximately 80 to 165 degrees, in this embodiment. The sheath 122 is sized and configured to be advanced over the shaft 103 of the lead 104 until a portion of its distal end 128 abuts the distal tip 118 of the lead 104, as illustrated in FIG. 19. Thus, the ball shaped tip 118 of this embodiment also prevents the sheath 122 from extending thereover. Passage of the sheath 122 over the lead 104 causes the lead 104 to bend in accordance with the precurvature of the sheath 122. Thus, the sheath 122 assists in steering the lead 104 along the spinal column S and toward a target DRG, such as in a lateral direction. It may be appreciated that the angle α may optionally be smaller, such as less than 80 degrees, forming a U-shape or tighter bend.

Referring back to FIG. 18C, an example embodiment of a stylet 124 is illustrated. In this embodiment, the stylet 124 has a distal end 130 which is pre-curved so that its radius of curvature is in the range of approximately 0.1 to 0.5 inches. The stylet 124 is sized and configured to be advanced within the stylet lumen 119 of the lead 104. Typically the stylet 124 extends therethrough so that its distal end 130 aligns with the distal end 101 of the lead 104. Passage of the stylet 124 through the lead 104 causes the lead 104 to bend in accordance with the precurvature of the stylet 124. Typically, the stylet 124 has a smaller radius of curvature, or a tighter bend, than the sheath 122. Therefore, as shown in FIG. 20, when the stylet 124 is disposed within the lead 104, extension of the lead 104 and stylet 124 through the sheath 122 bends or directs the lead 104 through a first curvature 123. Further extension of the lead 104 and stylet 124 beyond the distal end 128 of the sheath 122 allows the lead 104 to bend further along a second curvature 125. When approaching a target DRG, the second curvature allows the laterally directed lead 104 to now curve around toward the target DRG, such as along the nerve root angulation. This two step curvature allows the lead 104 to be successfully positioned so that at least one of the electrodes 106 is on, near, about, adjacent or in proximity to the target DRG, particularly by making a sharp turn along the angle θ. In addition, the electrodes 106 are spaced to assist in making such a sharp turn.

Thus, the lead 104 does not require stiff or torqueable construction since the lead 104 is typically not torqued or steered by itself The lead 104 is positioned with the use of the sheath 122 and stylet 124 which direct the lead 104 through the two step curvature. This eliminates the need for the operator to torque the lead 104 and optionally the sheath 122 with multiple hands. This also allows the lead 104 to have a lower profile and smaller diameter, as well as a very soft and flexible construction. This, in turn, minimizes erosion, irritation of the neural tissue and discomfort created by pressure on nerve tissue, such as the target DRG and/or other nerves, once the lead 104 is implanted. In addition, such a soft and flexible lead 104 will minimize the amount of force translated to the distal end of the lead 104 by body movement (e.g. flexion, extension, torsion).

Referring back to FIG. 18D, an embodiment of an introducing needle 126 is illustrated. The introducing needle 126 is used to access the epidural space of the spinal cord S. The needle 126 has a hollow shaft 127 and typically has a very slightly curved distal end 132. The shaft 127 is sized to allow passage of the lead 104, sheath 122 and stylet 124 therethrough. In some embodiments, the needle 126 is 14 gauge which is typically the size of epidural needles used to place conventional percutaneous leads within the epidural space. However, it may be appreciated that other sized needles may also be used, particularly smaller needles such as 15-18 gauge. Alternatively, non-standardized sized needles may be used.

The needle is atraumatic so as to not damage the sheath 122 when the sheath 122 is advanced or retracted. In some embodiments, the shaft 127 comprises a low friction material, such as bright hypotubing, made from bright steel (a product formed from the process of drawing hot rolled steel through a die to impart close dimensional tolerances, a bright, scale free surface and improved mechanical properties. Other materials include polytetrafluoroethylene (PTFE) impregnated or coated hypotubing. In addition, it may be appreciated that needles having various tips known to practitioners or custom tips designed for specific applications may also be used. The needle 126 also typically includes a luer fitting 134, such as a Luer-Lok™ fitting, or other fitting near its proximal end. The luer fitting 134 is a female fitting having a tabbed hub which engages threads in a sleeve on a male fitting, such as a syringe. The needle 126 may also have a luer fitting on a side port, so as to allow injection through the needle 126 while the sheath 122 is in the needle 126. In some embodiments, the luer fitting is tapered to allow for easier introduction of a curved sheath into the hollow shaft 127.

The above described delivery system 120 is used for epidural delivery of the lead 104 through the patient anatomy toward a target DRG or points beyond the foramen, such as the peripheral system. Thus, embodiments of epidural delivery methods of the present invention are described herein. In particular, such embodiments are described and illustrated as an antegrade approach. It may be appreciated that, alternatively, the devices and systems of the present invention may be used with a retrograde approach or a contralateral approach. Likewise, at least some of the devices and systems may be used with a transforaminal approach, wherein the target is approached from outside of the spinal column. Further, a target may be approached through the sacral hiatus or through a bony structure such as a pedicle, lamina or other structure.

Epidural delivery involves accessing the epidural space. The epidural space is accessed with the use of the introducing needle 126, as illustrated in FIG. 18D. Typically, the skin is infiltrated with local anesthetic such as lidocaine over the identified portion of the epidural space. The insertion point is usually near the midline M, although other approaches may be employed. Typically, the needle 126 is inserted to the ligamentum flavum and a loss of resistance to injection technique is used to identify the epidural space. A syringe 140 is then attached to the needle 126, typically containing air or saline. Traditionally either air or saline has been used for identifying the epidural space, depending on personal preference. When the tip of the needle 126 enters a space of negative or neutral pressure (such as the epidural space), there will be a “loss of resistance” and it will be possible to inject through the syringe 140. In addition to the loss of resistance technique, realtime observation of the advancing needle 126 may be achieved with a portable ultrasound scanner or with fluoroscopy. Likewise, a guidewire may be advanced through the needle 126 and observed within the epidural space with the use of fluoroscopy.

Once the needle 126 has been successfully inserted into the epidural space, the syringe 140 is removed. The stylet 124 is inserted into the lead 104 and the sheath 122 is advanced over the lead 104. The sheath 122 is positioned so that its distal end 128 is near or against the distal tip 106 of the lead 104 causing the lead 104 to follow the curvature of the sheath 122. The stylet 124, lead 104 and sheath 122 are then inserted through the needle 126, into the epidural space and the assembled sheath 122/lead 104/stylet 124 emerges therefrom. The rigidity of the needle 126 straightens the more flexible sheath 122 as it passes therethrough. However, upon emergence, the sheath 122 is allowed to bend along or toward its precurvature. In some embodiments, the shape memory of the sheath 122 material allows the sheath 122 to retain more than 50% of its precurved shape upon passing through the needle 126. Such bending assists in steering of the lead 104 within the epidural space. This is particularly useful when using a retrograde approach to navigate across the transition from the lumbar spine to the sacral spine. The sacrum creates a “shelf” that resists ease of passage into the sacrum. The precurved sheath 122 is able to more easily pass into the sacrum, reducing operating time and patient discomfort.

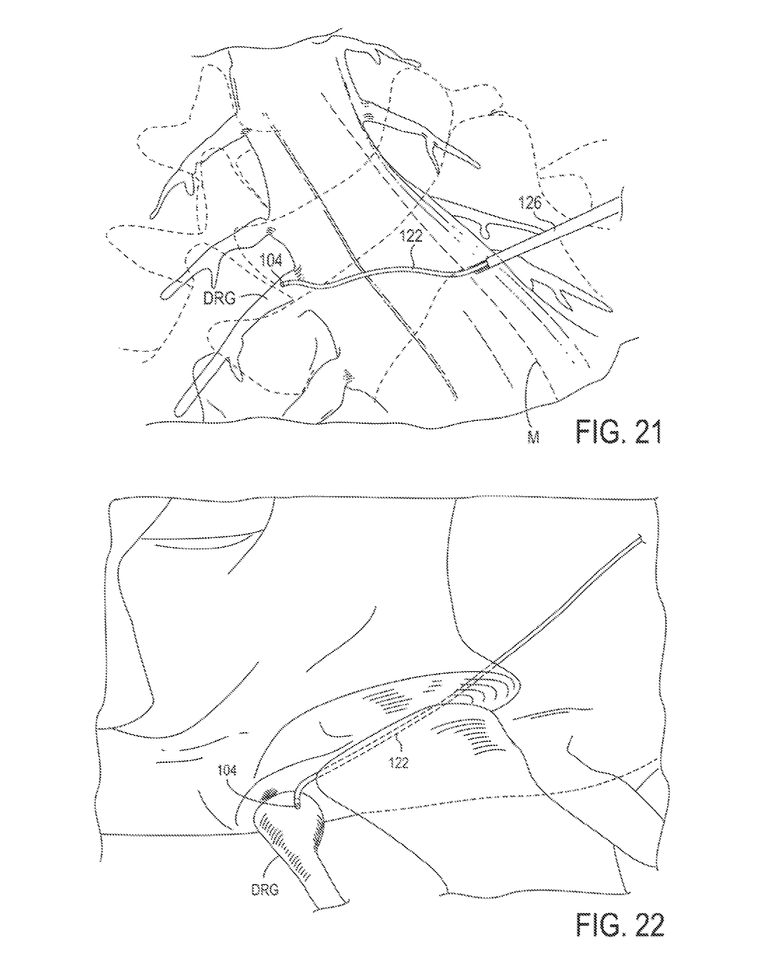

Referring to FIG. 21, the assembled sheath 122/lead 104/stylet 124 is advanced within the epidural space toward a DRG. Steering and manipulation is controlled proximally and is assisted by the construction of the assembled components and the precurvature of the sheath 122. In particular, the precurvature of the sheath 122 directs the lead 104 laterally outwardly, away from the midline M of the spinal column. FIG. 21 illustrates the assembled sheath 122/lead 104/stylet 124 advanced toward a DRG with the precurvature of the sheath 122 directing the lead 104 laterally outwardly.

Referring to FIG. 22, the lead 104/stylet 124 is then advanced beyond the distal end 128 of the sheath 122. In some embodiments, the lead 104 extends approximately 1-3 inches beyond the distal end 128 of the sheath 122. However, the lead 104 may extend any distance, such as less than 1 inch, 0.25-3 inches, or more than 3 inches. Likewise, the sheath 122 may be retracted to expose the lead 104, with or without advancement of the lead 104. This may be useful when advancement of the lead 104 is restricted, such as by compression of the foraminal opening. The curvature of the stylet 124 within the lead 104 causes the lead 104 to bend further, along this curvature. This allows the laterally directed lead 104 to now curve around toward the\DRG along the nerve root angulation. This two step curvature allows the lead 104 to be successfully steered to a desired position, such as having at least one of the electrodes 106 on, near or about the DRG. In addition, the ball shaped distal tip 118 resists trauma to the anatomy within the spinal column, such as the dural sac, ligaments, blood vessels, and resists imparting trauma to the DRG as the lead 104 is manipulated and advanced into place.

In some embodiments, the lead 104/stylet 124 is further advanced so that at least one of the electrodes 106 is positioned beyond the DRG. In some embodiments, the distal end of the lead 104/stylet 124 is extended through the foramen so as to position at least one electrode near the mixed spinal nerve SN, so as to selectively stimulate the mixed spinal nerve SN. In some embodiments, the lead 104/stylet 124 is advanced so that at least one electrode 106 is positioned along the medial branch MB of the dorsal ramus DRA. In other embodiments, the lead 104/stylet 124 is advanced further through the foramen so that at least one electrode 106 is positioned along a portion of the ventral ramus VRA. In fact, the lead 104/stylet 124 assembly may be advanced to any of the target anatomies described herein, wherein the stylet 124 assists in steering the lead 104. For example, when directing the lead 104 toward the medial branch MB, such movement is contrary to the natural advancement along the ventral ramus VRA. Thus, the curvature of the stylet 124 may be used to direct the lead 104 away from the ventral ramus VRA, along the medial branch MB. Other similar turns and contorsions through the anatomy to points along the peripheral nervous system may be navigated with the assistance of the internal stylet 124.

It may be appreciated that in some embodiments, the assembled sheath 122/lead 104/stylet 124 is advanced through the foramen to points therebeyond, such as along the peripheral nervous system. In such embodiments, the sheath 122 adds additional steering capabilities as described above. This may be of particular usefulness when reaching target anatomies at a greater distance from the foramen or at locations reachable through tortuous anatomy.

Once desirably positioned, the sheath 122 and stylet 124 are typically removed leaving the lead 104 in place. However, optionally, the stylet 124 may be left within the lead 104 to stabilize the lead 104, to assist in maintaining position and to resist migration. The target anatomy may then be stimulated by providing stimulation energy to the at least one electrode 106. It may be appreciated that multiple electrodes may be energized to stimulate the target anatomy or various anatomies at the same time. It may also be appreciated that the electrodes may be energized prior to removal of the stylet 124 and/or sheath 122, particularly to ascertain the desired positioning of the lead 104. It may further be appreciated that the sheath 122 may be retracted to expose the lead 104 rather than advancing the lead 104 therethrough.

Any number of leads 104 may be introduced through the same introducing needle 126. In some embodiments, the introducing needle 126 has more than one lumen, such as a double-barreled needle, to allow introduction of leads 100 through separate lumens. Further, any number of introducing needles 126 may be positioned along the spinal column for desired access to the epidural space. In some embodiments, a second needle is placed adjacent to a first needle. The second needle is used to deliver a second lead to a spinal level adjacent to the spinal level corresponding to the first needle. In some instances, there is a tract in the epidural space and the placement of a first lead may indicate that a second lead may be easily placed through the same tract. Thus, the second needle is placed so that the same epidural tract may be accessed. In other embodiments, a second needle is used to assist in stabilizing the tip of a sheath inserted through a first needle. In such embodiments, the second needle is positioned along the spinal column near the target anatomy. As the sheath is advanced, it may use the second needle to buttress against for stability or to assist in directing the sheath. This may be particularly useful when accessing a stenosed foramen which resists access. The proximal ends of the leads 104 are connected with an IPG which is typically implanted nearby.

### Example Treatment Conditions

The devices, systems, and methods of the present invention may be used to treat a variety of pain-related and non-pain related conditions. In particular, the devices, systems and methods may be used to treat the neurological diseases, disorders and stroke listed in Table 1. It may be appreciated that the present invention may also be used to treat other diseases, disorders and conditions.

TABLE 1
Neurological Diseases, Disorders and Stroke

Acute Disseminated Encephalomyelitis
ADHD
Adie's Pupil
Adie's Syndrome
Adrenoleukodystrophy
Agenesis of the Corpus Callosum
Agnosia
Aicardi Syndrome
Aicardi-Goutieres Syndrome Disorder
AIDS - Neurological Complications
Alexander Disease
Alpers' Disease
ALS (Amyotrophic Lateral Sclerosis)
Alternating Hemiplegia
Alzheimer's Disease
Anencephaly
Aneurysm
Angelman Syndrome
Angiomatosis
Anoxia
Antiphospholipid Syndrome
Aphasia
Apraxia
Arachnoid Cysts
Arachnoiditis
Arnold-Chiari Malformation
Arteriovenous Malformation
Asperger Syndrome
Ataxia
Ataxia Telangiectasia
Ataxias and Cerebellar or Spinocerebellar Degeneration
Atrial Fibrillation and Stroke
Attention Deficit-Hyperactivity Disorder
Autism
Autonomic Dysfunction
Back Pain
Barth Syndrome
Batten Disease
Becker's Myotonia
Behcet's Disease
Bell's Palsy
Benign Essential Blepharospasm
Benign Focal Amyotrophy
Benign Intracranial Hypertension
Bernhardt-Roth Syndrome
Binswanger's Disease
Blepharospasm
Bloch-Sulzberger Syndrome
Brachial Plexus Birth Injuries
Brachial Plexus Injuries
Bradbury-Eggleston Syndrome
Brain and Spinal Tumors
Brain Aneurysm
Brain Injury
Brown-Sequard Syndrome
Bulbospinal Muscular Atrophy
CADASIL
Canavan Disease
Carpal Tunnel Syndrome
Causalgia
Cavernomas
Cavernous Angioma
Cavernous Malformation
Central Cervical Cord Syndrome
Central Cord Syndrome
Central Pain Syndrome
Central Pontine Myelinolysis
Cephalic Disorders
Ceramidase Deficiency
Cerebellar Degeneration
Cerebellar Hypoplasia
Cerebral Aneurysm
Cerebral Arteriosclerosis
Cerebral Atrophy
Cerebral Beriberi
Cerebral Cavernous Malformation
Cerebral Gigantism
Cerebral Hypoxia
Cerebral Palsy
Cerebro-Oculo-Facio-Skeletal Syndrome (COFS)
Charcot-Marie-Tooth Disease
Chiari Malformation
Cholesterol Ester Storage Disease
Chorea
Choreoacanthocytosis
Chronic Inflammatory Demyelinating Polyneuropathy (CIDP)
Chronic Orthostatic Intolerance
Chronic Pain
Cockayne Syndrome Type II
Coffin Lowry Syndrome
Colpocephaly
Coma
Complex Regional Pain Syndrome
Congenital Facial Diplegia
Congenital Myasthenia
Congenital Myopathy
Congenital Vascular Cavernous Malformations
Corticobasal Degeneration
Cranial Arteritis
Craniosynostosis
Cree encephalitis
Creutzfeldt-Jakob Disease
Cumulative Trauma Disorders
Cushing's Syndrome
Cytomegalic Inclusion Body Disease
Cytomegalovirus Infection
Dancing Eyes-Dancing Feet Syndrome
Dandy-Walker Syndrome
Dawson Disease
De Morsier's Syndrome
Dejerine-Klumpke Palsy
Dementia
Dementia - Multi-Infarct
Dementia - Semantic
Dementia - Subcortical
Dementia With Lewy Bodies
Dentate Cerebellar Ataxia
Dentatorubral Atrophy
Dermatomyositis
Developmental Dyspraxia
Devic's Syndrome
Diabetic Neuropathy
Diffuse Sclerosis
Dravet Syndrome
Dysautonomia
Dysgraphia
Dyslexia
Dysphagia
Dyspraxia
Dyssynergia Cerebellaris Myoclonica
Dyssynergia Cerebellaris Progressiva
Dystonias
Early Infantile Epileptic Encephalopathy
Empty Sella Syndrome
Encephalitis
Encephalitis Lethargica
Encephaloceles
Encephalopathy
Encephalopathy (familial infantile)
Encephalotrigeminal Angiomatosis
Epilepsy
Epileptic Hemiplegia
Erb-Duchenne and Dejerine-Klumpke Palsies
Erb's Palsy
Essential Tremor
Extrapontine Myelinolysis
Fabry Disease
Fahr's Syndrome
Fainting
Familial Dysautonomia
Familial Hemangioma
Familial Idiopathic Basal Ganglia Calcification
Familial Periodic Paralyses
Familial Spastic Paralysis
Farber's Disease
Febrile Seizures
Fibromuscular Dysplasia
Fisher Syndrome
Floppy Infant Syndrome
Foot Drop
Friedreich's Ataxia
Frontotemporal Dementia
Gangliosidoses
Gaucher's Disease
Gerstmann's Syndrome
Gerstmann-Straussler-Scheinker Disease
Giant Axonal Neuropathy
Giant Cell Arteritis
Giant Cell Inclusion Disease
Globoid Cell Leukodystrophy
Glossopharyngeal Neuralgia
Glycogen Storage Disease
Guillain-Barré Syndrome
Hallervorden-Spatz Disease
Head Injury
Headache
Hemicrania Continua
Hemifacial Spasm
Hemiplegia Alterans
Hereditary Neuropathies
Hereditary Spastic Paraplegia
Heredopathia Atactica Polyneuritiformis
Herpes Zoster
Herpes Zoster Oticus
Hirayama Syndrome
Holmes-Adie syndrome
Holoprosencephaly
HTLV-1 Associated Myelopathy
Hughes Syndrome
Huntington's Disease
Hydranencephaly
Hydrocephalus
Hydrocephalus - Normal Pressure
Hydromyelia
Hypercortisolism
Hypersomnia
Hypertonia
Hypotonia
Hypoxia
Immune-Mediated Encephalomyelitis
Inclusion Body Myositis
Incontinentia Pigmenti
Infantile Hypotonia
Infantile Neuroaxonal Dystrophy
Infantile Phytanic Acid Storage Disease
Infantile Refsum Disease
Infantile Spasms
Inflammatory Myopathies
Iniencephaly
Intestinal Lipodystrophy
Intracranial Cysts
Intracranial Hypertension
Isaac's Syndrome
Joubert Syndrome
Kearns-Sayre Syndrome
Kennedy's Disease
Kinsbourne syndrome
Kleine-Levin Syndrome
Klippel-Feil Syndrome
Klippel-Trenaunay Syndrome (KTS)
Klüver-Bucy Syndrome
Korsakoff's Amnesic Syndrome
Krabbe Disease
Kugelberg-Welander Disease
Kuru
Lambert-Eaton Myasthenic Syndrome
Landau-Kleffner Syndrome
Lateral Femoral Cutaneous Nerve Entrapment
Lateral Medullary Syndrome
Learning Disabilities
Leigh's Disease
Lennox-Gastaut Syndrome
Lesch-Nyhan Syndrome
Leukodystrophy
Levine-Critchley Syndrome
Lewy Body Dementia
Lipid Storage Diseases
Lipoid Proteinosis
Lissencephaly
Locked-In Syndrome
Lou Gehrig's Disease
Lupus - Neurological Sequelae
Lyme Disease - Neurological Complications
Machado-Joseph Disease
Macrencephaly
Megalencephaly
Melkersson-Rosenthal Syndrome
Meningitis
Meningitis and Encephalitis
Menkes Disease
Meralgia Paresthetica
Metachromatic Leukodystrophy
Microcephaly
Migraine
Miller Fisher Syndrome
Mini Stroke
Mitochondrial Myopathies
Moebius Syndrome
Monomelic Amyotrophy
Motor Neuron Diseases
Moyamoya Disease
Mucolipidoses
Mucopolysaccharidoses
Multifocal Motor Neuropathy
Multi-Infarct Dementia
Multiple Sclerosis
Multiple System Atrophy
Multiple System Atrophy with Orthostatic Hypotension
Muscular Dystrophy
Myasthenia - Congenital
Myasthenia Gravis
Myelinoclastic Diffuse Sclerosis
Myoclonic Encephalopathy of Infants
Myoclonus
Myopathy
Myopathy - Congenital
Myopathy - Thyrotoxic
Myotonia
Myotonia Congenita
Narcolepsy
Neuroacanthocytosis
Neurodegeneration with Brain Iron Accumulation
Neurofibromatosis
Neuroleptic Malignant Syndrome
Neurological Complications of AIDS
Neurological Complications of Lyme Disease
Neurological Consequences of Cytomegalovirus Infection
Neurological Manifestations of Pompe Disease
Neurological Sequelae Of Lupus
Neuromyelitis Optica
Neuromyotonia
Neuronal Ceroid Lipofuscinosis
Neuronal Migration Disorders
Neuropathy - Hereditary
Neurosarcoidosis
Neurosyphilis
Neurotoxicity
Nevus Cavernosus
Niemann-Pick Disease
Normal Pressure Hydrocephalus
Occipital Neuralgia
Ohtahara Syndrome
Olivopontocerebellar Atrophy
Opsoclonus Myoclonus
Orthostatic Hypotension
O'Sullivan-McLeod Syndrome
Overuse Syndrome
Pain
Pantothenate Kinase-Associated Neurodegeneration
Paraneoplastic Syndromes
Parkinson's Disease
Paroxysmal Choreoathetosis
Paroxysmal Hemicrania
Parry-Romberg
Pelizaeus-Merzbacher Disease
Pena Shokeir II Syndrome
Perineural Cysts
Periodic Paralyses
Peripheral Neuropathy
Periventricular Leukomalacia
Persistent Vegetative State
Pervasive Developmental Disorders
Phytanic Acid Storage Disease
Pick's Disease
Pinched Nerve
Piriformis Syndrome
Pituitary Tumors
Polymyositis
Pompe Disease
Porencephaly
Postherpetic Neuralgia
Postinfectious Encephalomyelitis
Post-Polio Syndrome
Postural Hypotension
Postural Orthostatic Tachycardia Syndrome
Postural Tachycardia Syndrome
Primary Dentatum Atrophy
Primary Lateral Sclerosis
Primary Progressive Aphasia
Prion Diseases
Progressive Hemifacial Atrophy
Progressive Locomotor Ataxia
Progressive Multifocal Leukoencephalopathy
Progressive Sclerosing Poliodystrophy
Progressive Supranuclear Palsy
Prosopagnosia
Pseudo-Torch syndrome
Pseudotoxoplasmosis syndrome
Pseudotumor Cerebri
Ramsay Hunt Syndrome I
Ramsay Hunt Syndrome II
Rasmussen's Encephalitis
Reflex Sympathetic Dystrophy Syndrome
Refsum Disease
Refsum Disease - Infantile
Repetitive Motion Disorders
Repetitive Stress Injuries
Restless Legs Syndrome
Retrovirus-Associated Myelopathy
Rett Syndrome
Reye's Syndrome
Rheumatic Encephalitis
Riley-Day Syndrome
Sacral Nerve Root Cysts
Saint Vitus Dance
Salivary Gland Disease
Sandhoff Disease
Schilder's Disease
Schizencephaly
Seitelberger Disease
Seizure Disorder
Semantic Dementia
Septo-Optic Dysplasia
Severe Myoclonic Epilepsy of Infancy (SMEI)
Shaken Baby Syndrome
Shingles
Shy-Drager Syndrome
Sjögren's Syndrome
Sleep Apnea
Sleeping Sickness
Sotos Syndrome
Spasticity
Spina Bifida
Spinal Cord Infarction
Spinal Cord Injury
Spinal Cord Tumors
Spinal Muscular Atrophy
Spinocerebellar Atrophy
Spinocerebellar Degeneration
Steele-Richardson-Olszewski Syndrome
Stiff-Person Syndrome
Striatonigral Degeneration
Stroke
Sturge-Weber Syndrome
Subacute Sclerosing Panencephalitis
Subcortical Arteriosclerotic Encephalopathy
SUNCT Headache
Swallowing Disorders
Sydenham Chorea
Syncope
Syphilitic Spinal Sclerosis
Syringohydromyelia
Syringomyelia
Systemic Lupus Erythematosus
Tabes Dorsalis
Tardive Dyskinesia
Tarlov Cysts
Tay-Sachs Disease
Temporal Arteritis
Tethered Spinal Cord Syndrome
Thomsen's Myotonia
Thoracic Outlet Syndrome
Thyrotoxic Myopathy
Tic Douloureux
Todd's Paralysis
Tourette Syndrome
Transient Ischemic Attack
Transmissible Spongiform Encephalopathies
Transverse Myelitis
Traumatic Brain Injury
Tremor
Trigeminal Neuralgia
Tropical Spastic Paraparesis
Troyer Syndrome
Tuberous Sclerosis
Vascular Erectile Tumor
Vasculitis Syndromes of the Central and Peripheral Nervous
Systems
Von Economo's Disease
Von Hippel-Lindau Disease (VHL)
Von Recklinghausen's Disease
Wallenberg's Syndrome
Werdnig-Hoffman Disease
Wernicke-Korsakoff Syndrome
West Syndrome
Whiplash
Whipple's Disease
Williams Syndrome
Wilson's Disease
Wolman's Disease
X-Linked Spinal and Bulbar Muscular Atrophy
Zellweger Syndrome

It may also be appreciated that although the methods, systems and devices have been described and illustrated herein as using an epidural approach, other approaches may be used including extra-forminal wherein the DRG or various nerves are approached from outside of the spinal column, not crossing the midline of the spinal cord, and/or not entering the epidural space. It may also be appreciated that the DRG or various nerves may be approached through a pedicle. For example, a needle may be advanced through the pedicle and the lead delivered through the needle.

It may be appreciated that in some embodiments, the systems or devices deliver an agent or drug formulation to one or more target spinal anatomies, e.g., a dorsal root ganglion, mixed spinal nerve SN, ventral ramus VRA, dorsal ramus DRA, lateral branch LB, medial branch MB, intermediate branch IB, and/or rami communicantes (white ramus and/or gray ramus), and/or portions of the peripheral nervous system, to name a few. Example system, methods and devices for delivering the agent to a target, such as a dorsal root ganglion, are provided in U.S. patent application Ser. No. 13/309,429, entitled “Directed Delivery of Drugs to Spinal Anatomy for the Treatment of Pain”, incorporated herein by reference for all purposes. It may be appreciated that in some embodiments, the device, method and system is configured to enable direct and specific electrical stimulation of the target anatomy in combination with delivery of the agent. For example, in some embodiments, electrical stimulation is in a temporal pattern which is coordinated with a temporal pattern of delivery of the agent. In some embodiments, combined delivery of electrical stimulation and agent delivery results in one or more effects, including but not limited to, (i) synergistic action of the agent and electrical stimulation, (ii) an increase in the selectivity of an agent to target DRG cell bodies, (iii) targeted activation of an agent delivered to the DRG and (iv) differential enhancement of an agent to delivered target DRG cell bodies.

Although the foregoing invention has been described in some detail by way of illustration and example, for purposes of clarity and understanding, it will be obvious that various alternatives, modifications and equivalents may be used and the above description should not be taken as limiting in scope of the invention.

What is claimed is:

1. A method of treating pain in a patient comprising:
positioning a lead having at least one electrode so that at least one of the at least one electrodes is near a dorsal root ganglion associated with the pain; and
selecting at least one stimulation parameter so that energy is provided to the at least one of the at least one electrodes which results in selective stimulation of at least a portion of the dorsal root ganglion, wherein the selective stimulation causes a massaging sensation which treats the pain.

2. A method as in claim 1, wherein positioning the lead further comprises positioning the lead so that at least one of the at least one electrodes is near a mixed spinal nerve, a dorsal ramus and/or a ventral ramus associated with the dorsal root ganglion.

3. A method as in claim 2, wherein selecting at least one stimulation parameter further comprises selecting at least one stimulation parameter so that energy is provided to at least one of the electrodes which results in selective stimulation of the mixed spinal nerve, the dorsal ramus, and/or the ventral ramus.

4. A method as in claim 1, wherein the dorsal root ganglion is disposed on spinal level T10, T11, T12, L1, L2, L3, L4 or L5.

5. A method as in claim 4, wherein the dorsal root ganglion is disposed on spinal level L2 or L3.

6. A method as in claim 4, wherein the pain is associated with a thoracolumbar junction of the patient.

7. A method as in claim 4, wherein the pain is located in a low back of the patient.

8. A method as in claim 1, further comprising selecting at least one stimulation parameter so that the selective stimulation causes undesired muscle contraction prior to selecting the at least one stimulation parameter so that the selective stimulation causes the massaging sensation.

9. A method as in claim 1, wherein selecting at least one stimulation parameter comprises selecting a frequency of at least 16 Hz.

10. A method as in claim 9, wherein selecting at least one stimulation parameter comprises selecting a frequency in the range of approximately 20-50 Hz.

11. A method as in claim 10, wherein selecting at least one stimulation parameter comprises selecting a frequency in the range of approximately 20 Hz.

12. A method as in claim 9, further comprising selecting a frequency of less than or equal to 10 Hz so that a motor contraction is generated and then increasing the frequency to the frequency of at least 16 Hz to cause the massaging sensation.

13. A method of treating a condition of a patient comprising:
advancing a lead having at least one electrode into an epidural space in an antegrade direction, laterally away from a midline of a spinal cord and through a foramen so that at least one of the at least one electrodes is positioned near a peripheral nerve associated with the condition; and
selecting at least one stimulation parameter so that energy is provided to the at least one of the at least one electrodes which results in selective stimulation of at least a portion of the peripheral nerve, wherein the selective stimulation treats the condition.

14. A method as in claim 13, wherein the selective stimulation causes a massaging sensation.

15. A method as in claim 13, wherein the peripheral nerve comprises a mixed spinal nerve.

16. A method as in claim 13, wherein the peripheral nerve comprises a dorsal ramus.

17. A method as in claim 16, wherein the peripheral nerve comprises a lateral branch, a medial branch, or an intermediate branch.

18. A method as in claim 13, wherein the peripheral nerve comprises a ventral ramus.

19. A method as in claim 13, further comprising selecting at least one stimulation parameter so that energy is provided to at least one electrode which results in selective stimulation of at least a portion of a dorsal root ganglion disposed within the foramen.

20. A method as in claim 13, wherein the foramen is disposed on spinal level T10, T11, T12, L1, L2, L3, L4 or L5.

21. A method as in claim 20, wherein the foramen is disposed on spinal level L2 or L3.

22. A method as in claim 20, wherein the condition comprises pain associated with the thoracolumbar junction.

23. A method as in claim 20, wherein the condition comprises pain located in the low back.

24. A method as in claim 13, wherein selecting at least one stimulation parameter comprises selecting a frequency of at least 16 Hz.

25. A method as in claim 24, wherein selecting at least one stimulation parameter comprises selecting a frequency in the range of approximately 20-50 Hz.

26. A method as in claim 25, wherein selecting at least one stimulation parameter comprises selecting a frequency in the range of approximately 20 Hz.

27. A method as in claim 24, further comprising selecting a frequency of less than or equal to 10 Hz so that a motor contraction is generated and then increasing the frequency to the frequency of at least 16 Hz to treats the condition by generating a massaging sensation.
